(19) United States
(12) Patent Application Publication
(10) Pub. No.: US 2007/0037756 A1
(43) Pub. Date: Feb. 15, 2007

(54) Compositions and methods for the prevention and treatment of circulatory conditions

(75) Inventors: David T. Lau, Wayland, MA (US); John Zhang, Wayland, MA (US)

Correspondence Address: Peter G. Carroll;MEDLEN & CARROLL, LLP, Suite 350, 101 Howard Street, San Francisco, CA, 94105, US

(73) Assignee: LEADTREK, INC, Wayland, MA (US)

(21) Appl. No.: 11/202,452

(22) Filed: Aug. 12, 2005

(51) Int. Cl. A61K 31/7024; A61K 31/353; A61K 31/343

(52) U.S. Cl. 514/25; 514/456; 514/469

(57) ABSTRACT

The present invention relates to compositions and the prevention and treatment of circulatory conditions. In one embodiment, the present invention contemplates a compositions comprising anti-platelet aggregating activity. In another embodiment, the present invention contemplates methods to prevent or treat vascular or cardiovascular diseases. In another embodiment, the present invention relates to a method to prevent or treat cardiovascular disease by administering a glycerol saccharide derivative, an arylpolyphenol, a saccharide substituted with an arylpolyphenol, or a steroid substituted with an arylpolyphenol capable of reducing platelet aggregation. In one embodiment, the invention relates to a method of using the compounds capable of reducing platelet aggregation in order to prevent the recurrence of a heart attack by administration of the compounds to a subject after the subject has experienced a heart attack.

### FIELD OF INVENTION

The present invention relates to compositions and the prevention and treatment of circulatory conditions. In one embodiment, the present invention contemplates a compositions comprising anti-platelet aggregating activity. In another embodiment, the present invention contemplates methods to prevent or treat vascular or cardiovascular diseases. In another embodiment, the present invention relates to a method to prevent or treat cardiovascular disease by administering a glycerol saccharide derivative, an arylpolyphenol, a saccharide substituted with an arylpolyphenol, or a steroid substituted with an arylpolyphenol capable of reducing platelet aggregation. In one embodiment, the invention relates to a method of using the compounds capable of reducing platelet aggregation in order to prevent the recurrence of a heart attack by administration of the compounds to a subject after the subject has experienced a heart attack.

### BACKGROUND OF THE INVENTION

Heart fatigue is responsible for a vast majority of unexpected mortality. There are many individual etiologies responsible for the various cardiovascular diseases, however, aberrations in platelet aggregation is known to affect many seemingly unrelated cardiovascular disease states. Acute and chronic cardiovascular diseases may have abnormalities in platelet aggregation as a causative factor. For example, ischemia, angina, or acute myocardial infarction may be a result of platelet aggregation within the coronary arteries. Alternatively, peripheral arterial disease or hypertension may be a result of generalized platelet aggregation throughout the peripheral vascular tree.

Current approaches to prevent and treat some cardiovascular diseases have focused on platelet aggregation inhibitors. Aspirin, due to its low cost, is the most commonly prescribed drug following heart attack. However, about 10-20% of patients cannot tolerate aspirin because of side effects. Lanas et al., “Treatment And Prevention Of Aspirin-Induced Gastroduodenal Ulcers And Gastrointestinal Bleeding” Expert Opin Drug Saf 1:245-252 (2002). Presumably, its anti-coagulant effect prevents additional platelet aggregation in the coronary arteries thereby protecting the heart from further ischemia. Because aspirin has gastrointestinal hemorrhagic consequences, however, it may not be the most desirable drug available. Thus, there is a need to identify therapeutics capable of preventing platelet aggregation that does not disrupt a subject's digestive operation (e.g., bleeding and ulcers).

Consequently, newer anti-coagulant drugs are now prescribed under the assumption that these drugs, while having similar clinical benefit, are not likely to induce gastrointestinal damage. Kulbertus H., “Aspirin: Recent Advances In Cardiovascular Prevention” Rev Med Liege. 59:695-703 (2004). Plavix™ (Clopidogrel) has been introduced as one possible alternative to aspirin. Fork “Gastroduodenal Tolerance Of 75 mg Clopidogrel Versus 325 mg Aspirin In Healthy Volunteers” Scand J Gastroenoterol 35:464-469 (2000). While studies indicate that Plavix™ is as effective as aspirin, many of those skilled in the art are beginning to doubt its safety. Recent, studies suggest that acquired haemophilia A may be associated with clopidogrel. Haj et al., BMJ. 329(7461):323 (2004). Additionally, while clopidogrel may not induce gastrointestinal damage to otherwise healthy tissue, there is evidence that they are more likely to result in the recurrence of ulceration previously caused by aspirin administration. Chan et al., “Clopidogrel Versus Aspirin And Esomeprazole To Prevent Recurrent Ulcer Bleeding” N Engl J. Med. 352:238-44 (2005). Significant side effects such as skin rashes, neutropenia and thrombotic thrombocytopenic purpura have been reported. Kam et al., “The Thienopyridine Derivatives (Platelet Adenosine Diphosphate Receptor Antagonists), Pharmacology And Clinical Developments” Anaesthesia 58:28-35 (2003).

What are needed in the art are novel compositions and methods that provide improved anti-platelet aggregation activity without the adverse effects associated with similar compounds currently being administered to patients.

### BRIEF SUMMARY OF THE INVENTION

The present invention relates to compositions and the prevention and treatment of circulatory conditions. In one embodiment, the present invention contemplates a compositions comprising anti-platelet aggregating activity. In another embodiment, the present invention contemplates methods to prevent or treat vascular or cardiovascular diseases. In another embodiment, the present invention relates to a method to prevent or treat cardiovascular disease by administering a glycerol saccharide derivative, an arylpolyphenol, a saccharide substituted with an arylpolyphenol, or a steroid substituted with an arylpolyphenol capable of reducing platelet aggregation. In one embodiment, the invention relates to a method of using the compounds capable of reducing platelet aggregation in order to prevent the recurrence of a heart attack by administration of the compounds to a subject after the subject has experienced a heart attack.

In some embodiments, the invention relates to a method of treating or preventing cardiovascular disease comprising administering to a subject a composition comprising a compound i) functioning to prevent platelet aggregation and ii) having a substituted or unsubstituted formula selected from the group consisting of:

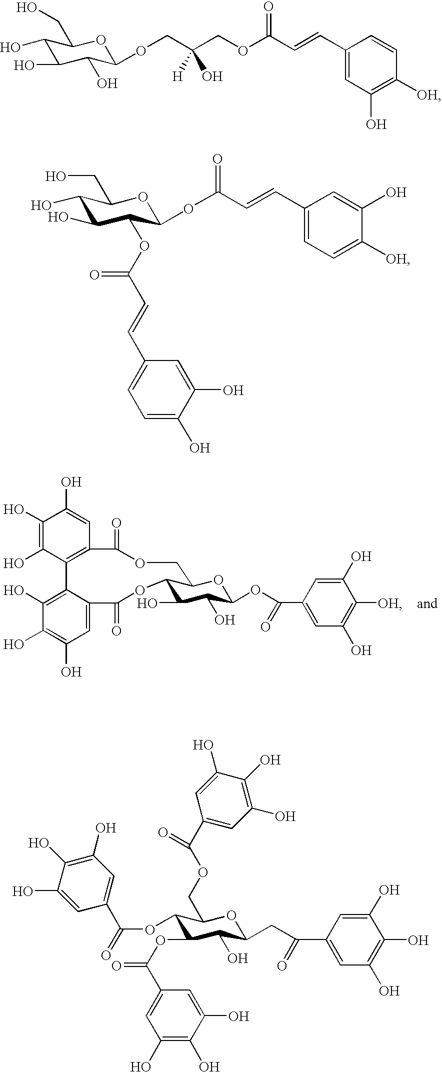

In some embodiments, the invention relates to a method of treating or preventing cardiovascular disease comprising administering to a subject a composition comprising a compound i) functioning to prevent platelet aggregation and ii) having the following substituted or unsubstituted formula: 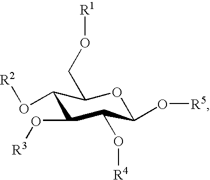
wherein R¹, R², R³, R⁴ and R⁵ are the same or different and independently are selected from the group consisting of: hydrogen, 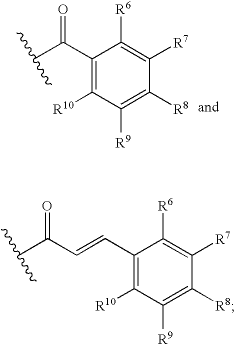
wherein R⁶, R⁷, R⁸, R⁹ and R¹⁰ are the same or different and independently hydrogen or hydroxyl; or R³, R⁴ and R⁵ are the same or different and independently are selected from the group consisting of: hydrogen and 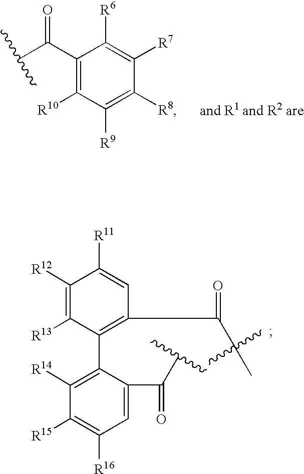
provided that of the group R¹, R², R³, R⁴ and R⁵ no more than three of the members are hydrogen.

In some embodiments, the invention relates to a method of treating or preventing cardiovascular disease comprising administering to a subject a composition comprising a compound i) functioning to prevent platelet aggregation and ii) having a substituted or unsubstituted formula selected from the group consisting of:

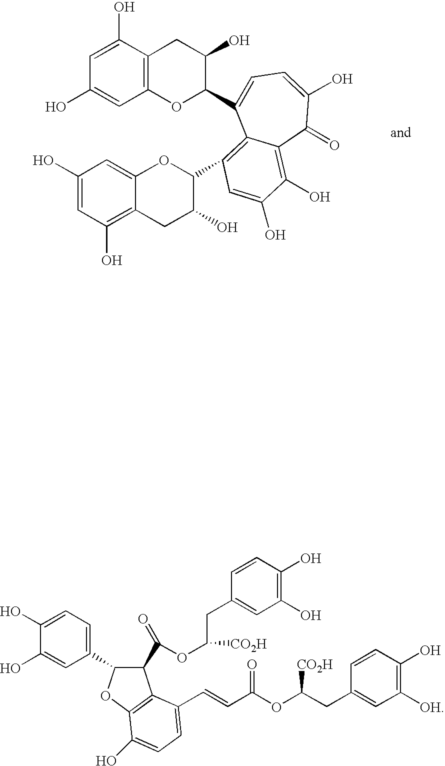

In some embodiments, the present invention contemplates the use of a compound functioning to reduce platelet aggregation for the manufacture of a medicament for the treatment of cardiovascular disease, arterial disease, or hypertension preferably ischemia, angina, acute myocardial infarction, more preferably those that do not cause adverse drug reactions in a subject.

In another embodiment, the present invention relates to compositions comprising a compound having the following structure: 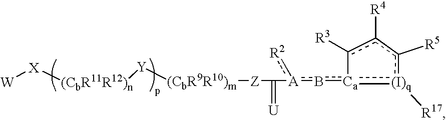
including stereoisomers, prodrugs and pharmaceutically acceptable salts thereof, wherein [symbol] is each individually and independently a single or double bond; A, B, and D are the same or different and independently carbon or nitrogen; Cₐ is carbon or nitrogen; C_{b} is carbon; E and F are the same or different and independently carbon, nitrogen, sulfur or oxygen; I_{q} is carbon, nitrogen, sulfur or oxygen, wherein q denotes an integer from 1 to 3 of I_{q} atom(s) the same or different and independently connected by single or double bonds groups forming a ring that includes Cₐ, D, E and F resulting in an aromatic carbocycle, heterocycle or homocycle; U, X, and Z are the same or different and independently oxygen, sulfur or NR¹⁸; W is a saccharide; Y is oxygen, sulfur, NR¹⁸, NR¹⁸, or C_{b}R¹¹OR¹⁹; R², R³, R⁸, are the same or different and independently hydrogen, alkyl, substituted alkyl, or absent, or R⁸ is hydrogen, alkyl, substituted alkyl, or absent, and R² and R³ are the same or different and independently carbon, oxygen, nitrogen, or sulfur taken together with the atom to which they are bonded form a six membered ring that includes A, B, Cₐ, and D resulting in an aromatic carbocycle, heterocycle or homocycle or R⁸ is hydrogen, alkyl, substituted alkyl, or absent, and R² and R³ are the same or different independently carbon, oxygen, nitrogen, or sulfur atom taken together with the atoms to which it is bonded form a five membered ring that includes A, B, Cₐ, and D resulting in an aromatic carbocycle, heterocycle or homocycle or R² is hydrogen, alkyl, substituted alkyl, or absent, and R⁸ and R³ are the same or different and independently carbon, oxygen, nitrogen, or sulfur taken together with the atom to which they are bonded form a five membered ring that includes B, Cₐ, and D resulting in an aromatic carbocycle, heterocycle or homocycle or R³ is hydrogen, alkyl, substituted alkyl, or absent, and R² and R⁸ is taken together with the atom to which they are bonded form a three, five or six membered ring of the same or different independently carbon, nitrogen, oxygen, or sulfur atoms that includes A and B resulting in an aromatic carbocycle, heterocycle or homocycle; R⁴, R⁵, and R¹⁷, are the same or different and independently hydrogen, halogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, as well as —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ, wherein Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl, substituted heterocyclealkyl, or absent; R⁹, R¹⁰, R¹¹, R¹², R¹⁸, R¹⁹, are the same or different and independently hydrogen, alkyl, or substituted alkyl; and n, m, and p are the same or different and independently 1 to 20, preferably 1 to 2.

In another embodiment, the present invention is a compound having the following formula: 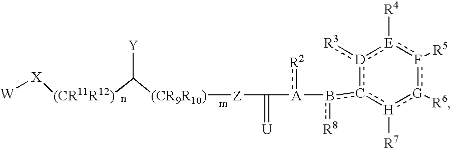
including stereoisomers, prodrugs and pharmaceutically acceptable salts thereof, wherein [symbol] is the same or different and independently a single or double bond; A, B, and D are the same or different and independently a carbon or nitrogen; C is carbon; E, F, G, and H are the same or different and independently carbon, nitrogen, sulfur or oxygen; U, X, and Z is the same or different and independently oxygen, sulfur or NR¹⁸; W is a saccharide; Y is hydrogen, hydroxy, acyloxy, SH, NHR¹⁸, NR¹⁸R¹⁹ or OCOR¹⁹; R², R³, R⁸, are the same or different and independently hydrogen, alkyl, substituted alkyl, or absent, or R⁸ is hydrogen, alkyl, substituted alkyl, or absent, and R² and R³ are the same or different and independently carbon, oxygen, nitrogen, or sulfur taken together with the atom to which they are bonded form a six membered ring that includes A, B, Cₐ, and D resulting in an aromatic carbocycle, heterocycle or homocycle or R⁸ is hydrogen, alkyl, substituted alkyl, or absent, and R² and R³ are the same carbon, oxygen, nitrogen, or sulfur atom taken together with the atoms to which it is bonded form a five membered ring that includes A, B, Cₐ, and D resulting in an aromatic carbocycle, heterocycle or homocycle or R² is hydrogen, alkyl, substituted alkyl, or absent, and R⁸ and R³ are the same or different and independently carbon, oxygen, nitrogen, or sulfur taken together with the atom to which they are bonded form a five membered ring that includes B, Cₐ, and D resulting in an aromatic carbocycle, heterocycle or homocycle or R³ is hydrogen, alkyl, substituted alkyl, or absent, and R² and R⁸ is taken together with the atom to which they are bonded form a three, five or six membered ring of the same or different independently carbon, nitrogen, oxygen, or sulfur atoms that includes A and B resulting in an aromatic carbocycle, heterocycle or homocycle; R⁴, R⁵, R⁶, and R⁷, are the same or different and independently hydrogen, alkyl, substituted alkyl, alkoxy, substituted alkoxy or absent; R⁹, R¹⁰, R¹¹, R¹², R¹⁸, R¹⁹, are the same or different and independently hydrogen, alkyl, or substituted alkyl; and n and m are the same or different and independently 1 to 20, preferably 1 to 2.

In another embodiment, the present invention is a compound having the following formula: 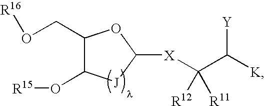
including stereoisomers, prodrugs and pharmaceutically acceptable salts thereof, wherein K is 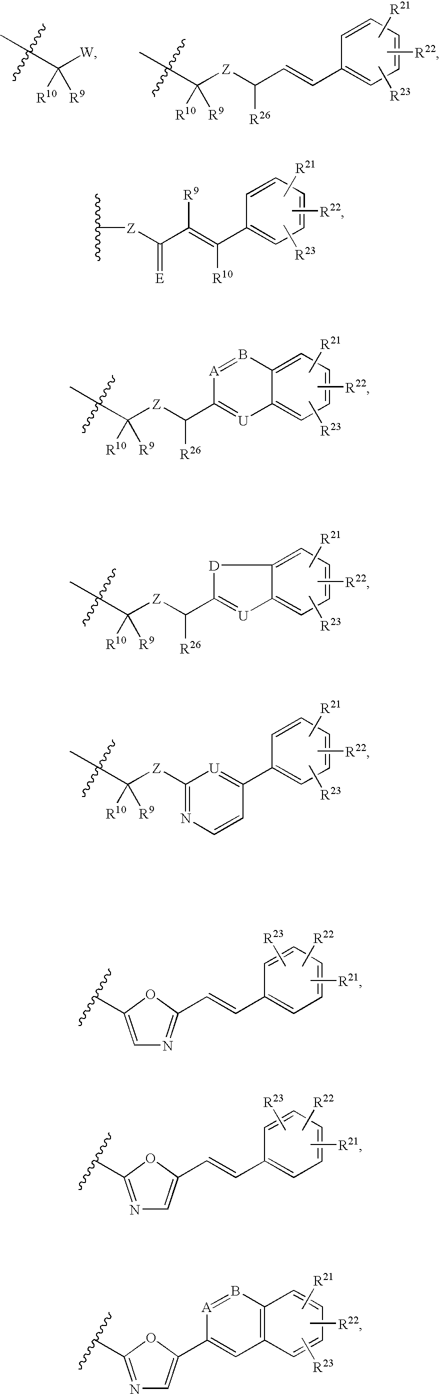 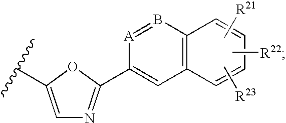
A, B, and U are carbon or nitrogen; E is oxygen or sulfur; D is carbon, nitrogen, oxygen, or sulfur; W is hydroxy, alkoxy, substituted alkoxy, acyloxy or substituted acyloxy; X and Z are the same or different and independently oxygen, sulfur, CH₂, or NR¹⁸; Y is hydrogen, hydroxy, —SH, —NHR¹⁹, —NR¹⁹R²⁰, —CH₂OR²⁴, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl or Y and X taken together with the atoms to which they are bonded form a five or six membered ring where X is nitrogen; J_{λ} is —CHOR¹⁴—, wherein _{λ} denotes an integer from 1 to 3 of J_{λ} groups the same or different and independently sequentially connected together with the atoms that they are attached to from a five, six, or seven membered ring; R²¹, R²², R²³, and R²⁴ are the same or differently and independently hydrogen, halogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ; Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; R⁹, R¹⁰, R¹¹, R¹², R¹⁸ R¹⁹ and R²⁰ are the same or different and independently hydrogen or alkyl; R²⁶ is hydrogen or alkyl; and R¹⁴, R¹⁵, and R¹⁶ are the same or different and independently hydrogen, alkyl, substituted alkyl, acyl or substituted acyl.

In another embodiment, the present invention is a compound having the following formula: 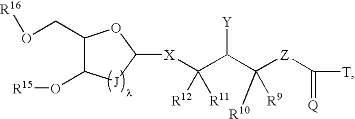
including stereoisomers, prodrugs and pharmaceutically acceptable salts thereof, wherein Q is oxygen, nitrogen, sulfur, two hydrogens, or CH₂; X and Z are the same or different and independently oxygen, sulfur or NR¹⁸; Y is hydrogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl; T is 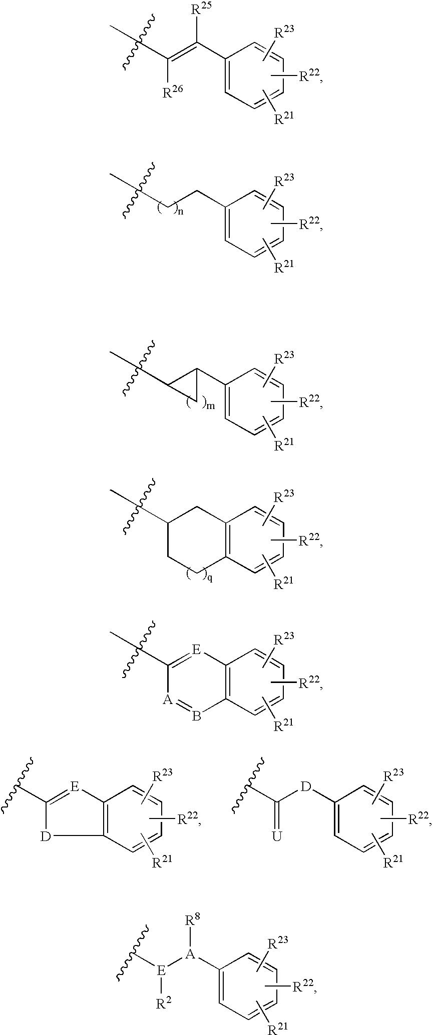
substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ; A, B, and E, is the same or differently and independently carbon or nitrogen, D is carbon, nitrogen, oxygen or sulfur; U is oxygen, sulfur, CH₂, NR¹⁸, or two hydrogens; n is 1 to 6, preferably 1 to 2; m is 1 to 5; q is 1 or 2; J_{λ} is —CHOR¹⁴—, wherein _{λ} denotes an integer from 1 to 3 of J_{λ} groups the same or different and independently sequentially connected together with the atoms that they are attached to from a five, six, or seven membered ring; R⁹, R¹⁰, R¹¹, R¹², and R¹⁸ are the same or different and independently hydrogen or alkyl; R¹⁴, R¹⁵, and R¹⁶ are the same or different and independently hydrogen, alkyl, substituted alkyl, acyl or substituted acyl; R²¹, R²², and R²³ are the same or differently and independently hydrogen, halogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ; Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; and R²⁵ and R²⁶ is the same or differently and independently hydrogen or alkyl.

In another embodiment, the present invention is a compound having the following formula: 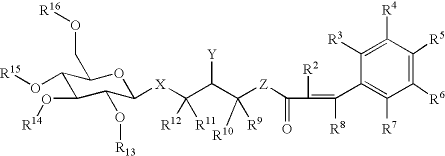
including stereoisomers, prodrugs and pharmaceutically acceptable salts thereof, wherein X and Z are the same or different and independently oxygen, sulfur or NR¹⁸; Y is hydrogen, hydroxy, SH, or NHR¹; R², R⁸ are the same or different and independently hydrogen, alkyl, or substituted alkyl; R³, R⁵, R⁵, R⁶, and R⁷ are the same or different and independently hydrogen, alkyl, substituted alkyl, alkoxy, or substituted alkoxy; R⁹, R¹⁰, R¹¹, R¹², and R¹⁸ are the same or different and independently hydrogen or alkyl; and R¹, R¹³, R¹⁴, R¹⁵, and R¹⁶ are the same or different and independently hydrogen, alkyl, substituted alkyl, acyl or substituted acyl.

In another embodiment, present invention is a substituted or unsubstituted compound having the following formula: 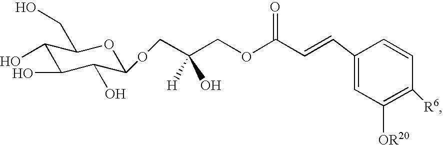
including, prodrugs and pharmaceutically acceptable salts thereof, wherein R⁶ is hydrogen or hydroxy and R²⁰ is hydrogen, alkyl, or acyl.

In another embodiment, the invention relates to a pharmaceutical composition for treating or preventing cardiovascular disorders comprising a compound selected from the group consisting of:

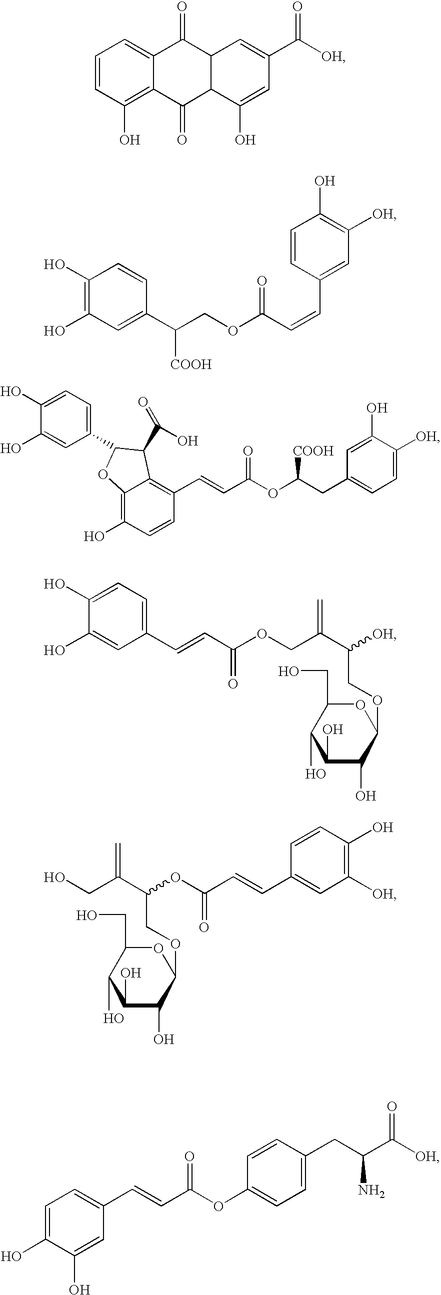

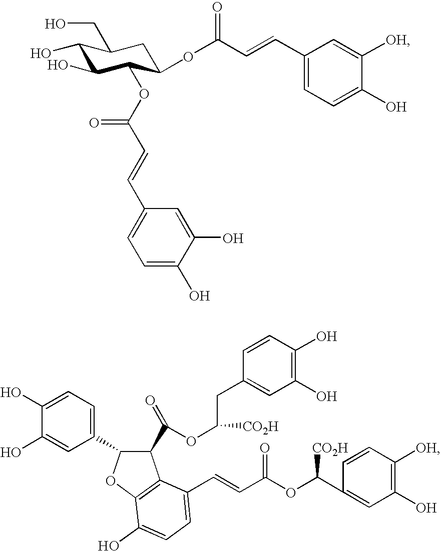

In another embodiment, the invention relates to a pharmaceutical composition for treating or preventing cardiovascular disorders comprising a compound selected from the group consisting of:

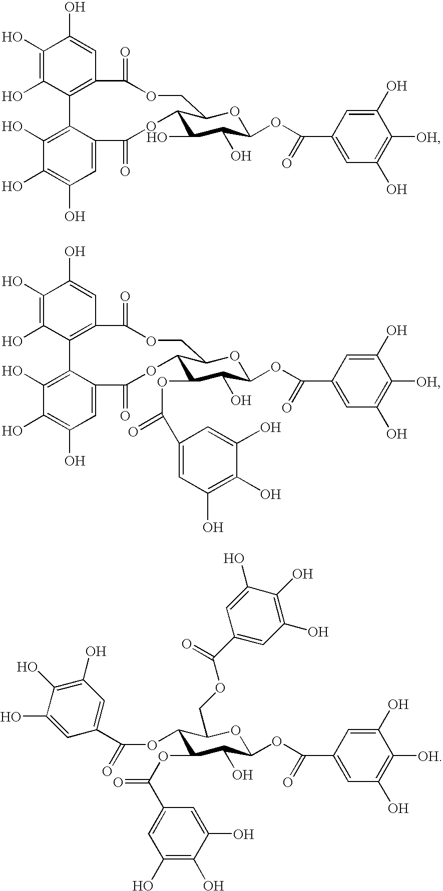

In another embodiment, the invention relates to a pharmaceutical composition for treating or preventing cardiovascular disorders comprising a compound selected from the group consisting of Phlo-OH, ProcB, WOG-Na, GK-1, GK-2, BJ-842, BJ-83, BJ-7, BJW-12C4, BJ-32, Caf Tryosi, SB, BJW-22C2, BJA 3226, BJW-24, BJ-821, BJA-3113322, rhein, RA, LA, Bai-Na, TF, BJ-851, T2-1, and T2-2.

In another embodiment the present invention contemplates a composition comprising a compound i) functioning to reduce platelet aggregation and ii) having a substituted structure of the following formula:

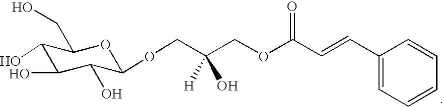

In another embodiment, the invention relates to compounds provided in FIGS. 13A through 13E.

In another embodiment, the present invention contemplates one or more of the compound(s) themselves. In further embodiments, the present invention contemplates compositions comprising the disclosed compounds, wherein one or more of the compound(s) by weight are greater that 1%, 5%, 15%, 20%, 25%, 30%, 40%, 45%, 50%, 55%, 60%, 65%, 70%, 75%, 80%, 85%, 90%, 95%, 98%, 99%, 99.5%, 99.99% of the entire composition, or as a component in a glycerol saccharide derivative composition.

### BRIEF DESCRIPTION OF THE DRAWINGS

FIG. 1. Illustration of methods for the preparation of derivatives.

FIG. 2. Illustration of methods for the preparation of derivatives.

FIG. 3. Illustration of methods for the preparation of derivatives.

FIG. 4. Illustration of methods for the preparation of derivatives.

FIG. 5. Illustration of methods for the preparation of derivatives.

FIG. 6. Illustration of methods for the preparation of derivatives.

FIG. 7. Illustration of identifying BJ-851 as preferred embodiment having an IC₅₀ of 2.94 μM in the assay of in Vitro Anti-platelet Aggregation.

FIG. 8. Illustration of identifying BJW-22C2 as preferred embodiment having an IC₅₀ of 0.62 μM in the assay of in Vitro Anti-platelet Aggregation.

FIG. 9. Illustration of identifying TF as preferred embodiment having an IC₅₀ of 4.75 μM in the assay of in Vitro Anti-platelet Aggregation.

FIG. 10. Illustration of identifying BJA 3226 as preferred embodiment having an IC₅₀ of 0.018 μM in the assay of in Vitro Anti-platelet Aggregation.

FIG. 11. Illustration of identifying SB as preferred embodiment having an IC₅₀ of 5.9 μM in the assay of in Vitro Anti-platelet Aggregation.

FIG. 12. A bar graph showing In Vivo Assay results For Anti-platelet Aggregation Activity.

FIG. 13A-E. Illustration of chemical embodiments.

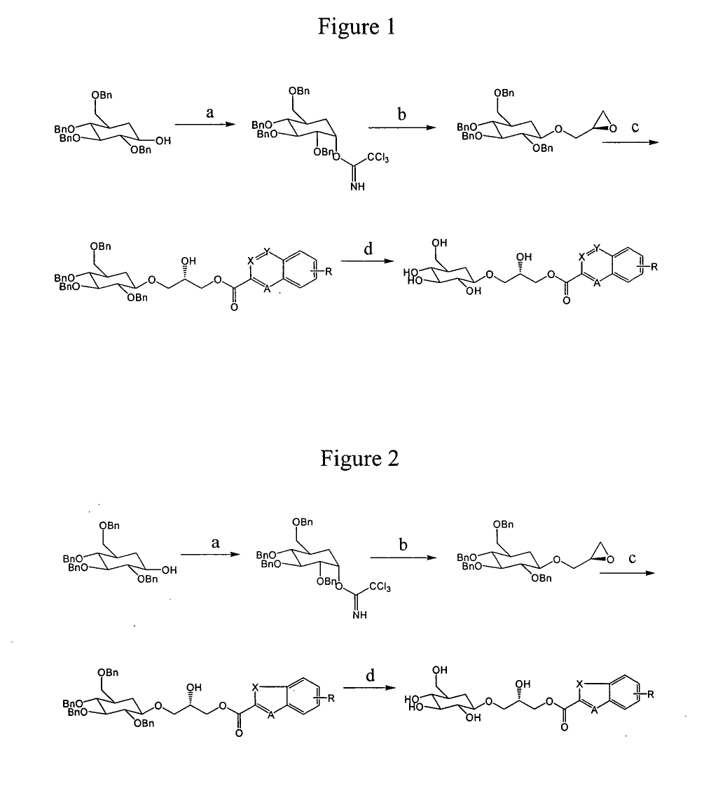

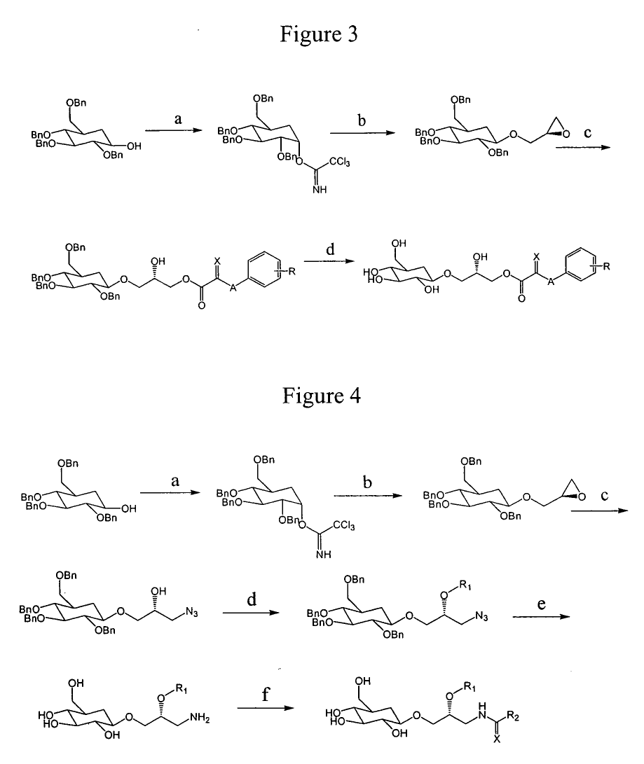

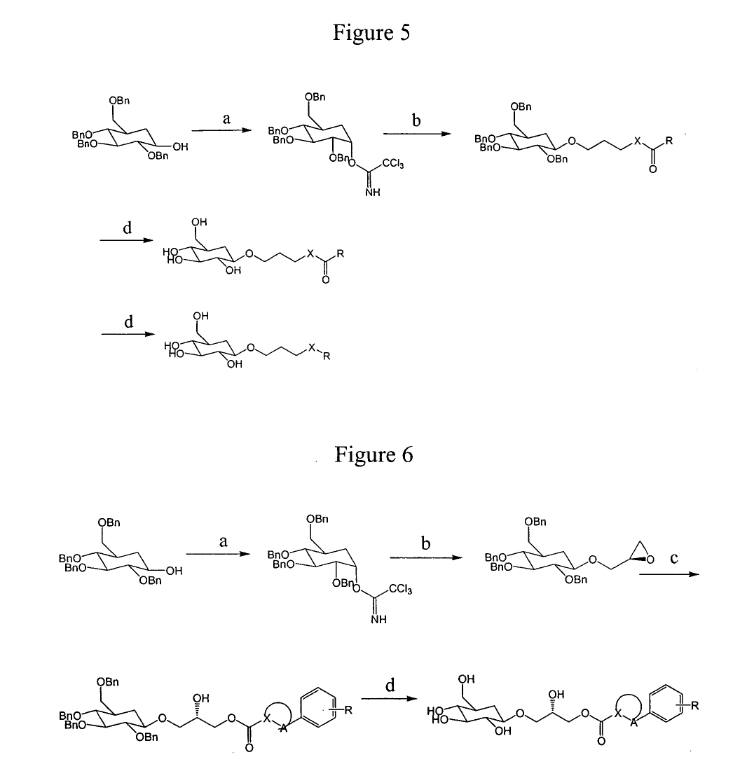

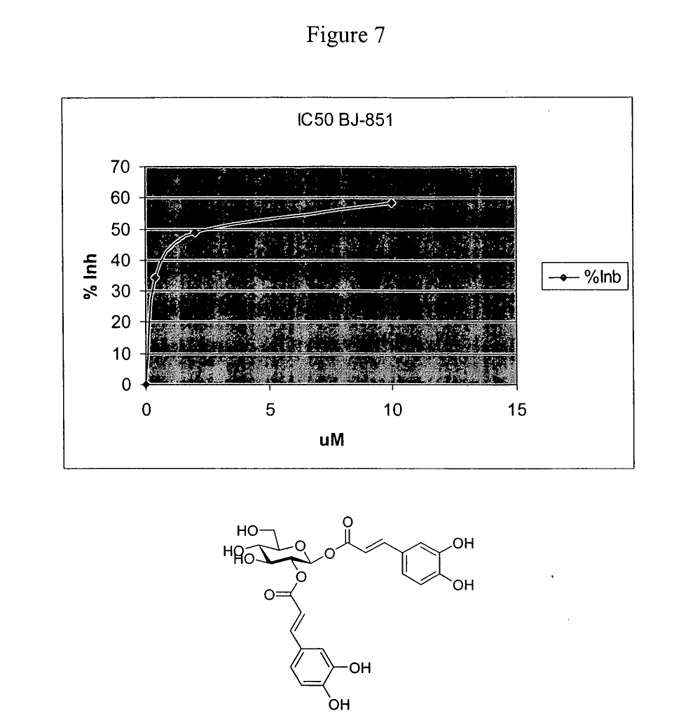

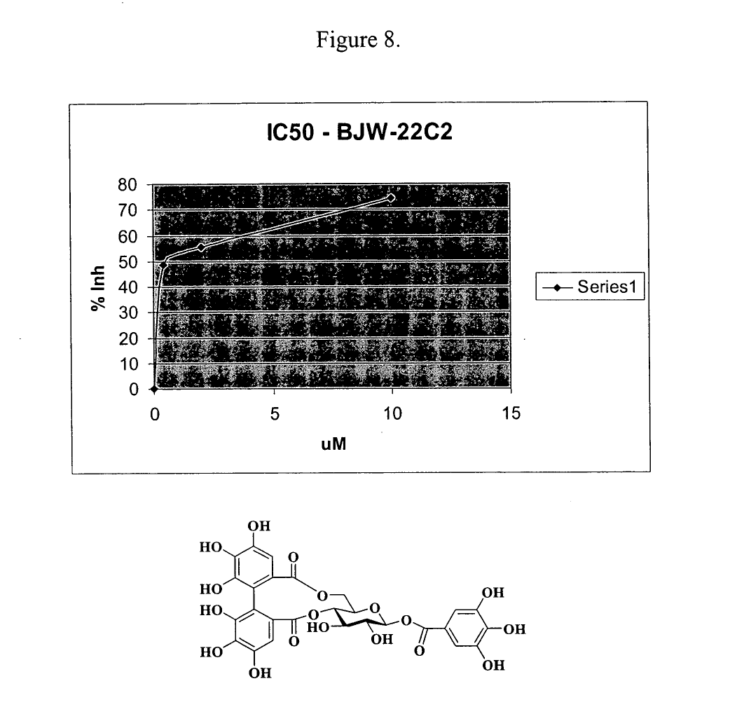

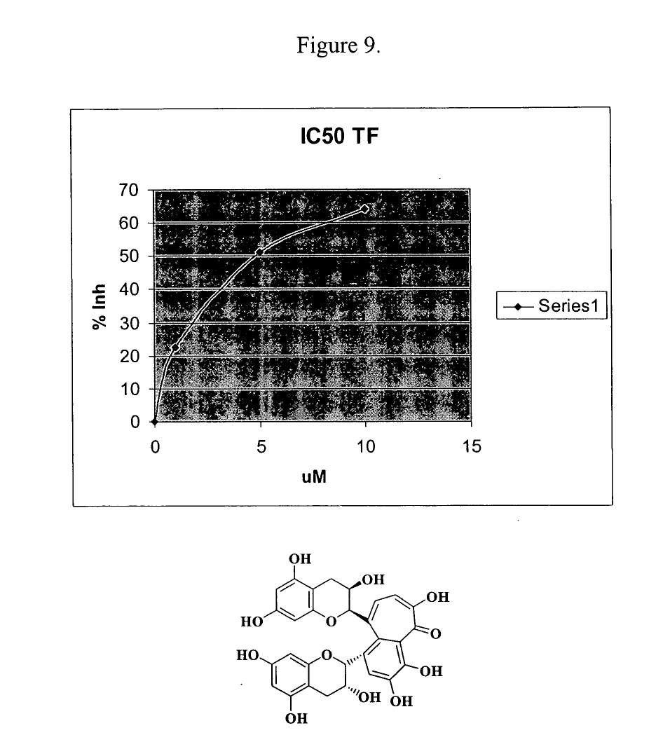

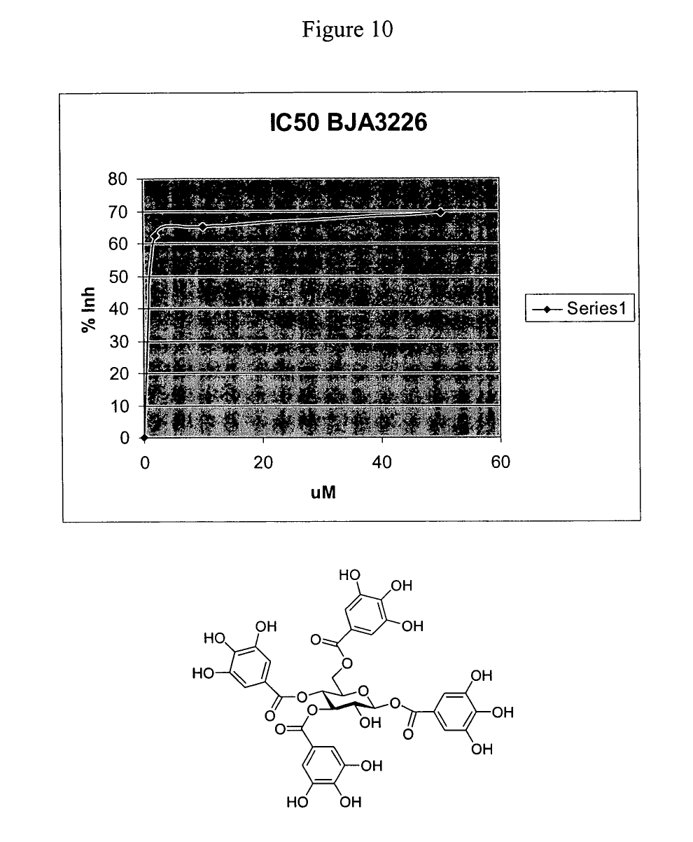

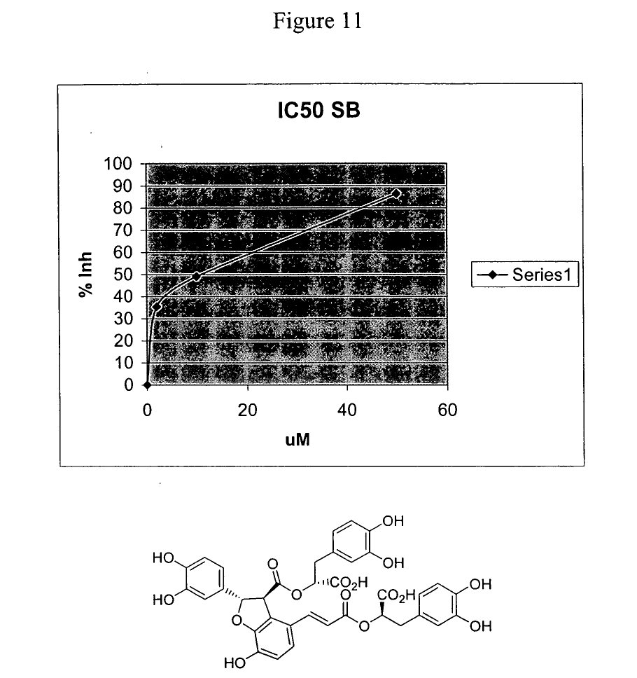

### DETAILED DESCRIPTION OF THE INVENTION

The present invention relates to compositions and the prevention and treatment of circulatory conditions. In one embodiment, the present invention contemplates a compositions comprising anti-platelet aggregating activity. In another embodiment, the present invention contemplates methods to prevent or treat vascular or cardiovascular diseases. In another embodiment, the present invention relates to a method to prevent or treat cardiovascular disease by administering a glycerol saccharide derivative, an arylpolyphenol, a saccharide substituted with an arylpolyphenol, or a steroid substituted with an arylpolyphenol capable of reducing platelet aggregation. In one embodiment, the invention relates to a method of using the compounds capable of reducing platelet aggregation in order to prevent the recurrence of a heart attack by administration of the compounds to a subject after the subject has experienced a heart attack.

In another embodiments, the present invention contemplates the use of a compound functioning to reduce platelet aggregation for the manufacture of a medicament for the treatment of cardiovascular disease, arterial disease, or hypertension preferably ischemia, angina, acute myocardial infarction, more preferably those that do not cause adverse drug reactions in a subject. In another embodiment, the present invention includes compositions comprising a compound having the following structure: 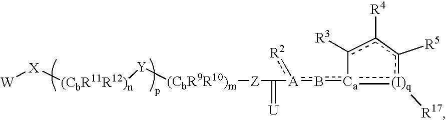
including stereoisomers, prodrugs and pharmaceutically acceptable salts thereof, wherein [symbol] is each individually and independently a single or double bond; A, B, and D are the same or different and independently carbon or nitrogen; Cₐ is carbon or nitrogen; C_{b} is carbon; E and F are the same or different and independently carbon, nitrogen, sulfur or oxygen; I_{q} is carbon, nitrogen, sulfur or oxygen, wherein q denotes an integer from 1 to 3 of I_{q} atom(s) the same or different and independently connected by single or double bonds groups forming a ring that includes Cₐ, D, E and F resulting in an aromatic carbocycle, heterocycle or homocycle; U, X, and Z are the same or different and independently oxygen, sulfur or NR¹⁸; W is a saccharide; Y is oxygen, sulfur, NR¹⁸, NR¹⁸, or C_{b}R¹¹OR¹⁹; R², R³, R⁸, are the same or different and independently hydrogen, alkyl, substituted alkyl, or absent, or R⁸ is hydrogen, alkyl, substituted alkyl, or absent, and R² and R³ are the same or different and independently carbon, oxygen, nitrogen, or sulfur taken together with the atom to which they are bonded form a six membered ring that includes A, B, Cₐ, and D resulting in an aromatic carbocycle, heterocycle or homocycle or R⁸ is hydrogen, alkyl, substituted alkyl, or absent, and R² and R³ are the same or different independently carbon, oxygen, nitrogen, or sulfur atom taken together with the atoms to which it is bonded form a five membered ring that includes A, B, Cₐ, and D resulting in an aromatic carbocycle, heterocycle or homocycle or R² is hydrogen, alkyl, substituted alkyl, or absent, and R⁸ and R³ are the same or different and independently carbon, oxygen, nitrogen, or sulfur taken together with the atom to which they are bonded form a five membered ring that includes B, Cₐ, and D resulting in an aromatic carbocycle, heterocycle or homocycle or R³ is hydrogen, alkyl, substituted alkyl, or absent, and R² and R⁸ is taken together with the atom to which they are bonded form a three, five or six membered ring of the same or different independently carbon, nitrogen, oxygen, or sulfur atoms that includes A and B resulting in an aromatic carbocycle, heterocycle or homocycle; R⁴, R⁵, and R¹⁷, are the same or different and independently hydrogen, halogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, as well as —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ, wherein Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl, substituted heterocyclealkyl, or absent; R⁹, R¹⁰, R¹¹, R¹², R¹⁸, R¹⁹, are the same or different and independently hydrogen, alkyl, or substituted alkyl; and n, m, and p are the same or different and independently 1 to 20, preferably 1 to 2.

In another embodiment, the present invention is a compound having the following formula: 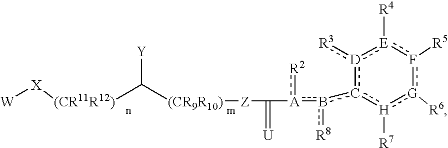
including stereoisomers, prodrugs and pharmaceutically acceptable salts thereof, wherein [symbol] is the same or different and independently a single or double bond; A, B, and D are the same or different and independently a carbon or nitrogen; C is carbon; E, F, G, and H are the same or different and independently carbon, nitrogen, sulfur or oxygen; U, X, and Z is the same or different and independently oxygen, sulfur or NR¹⁸; W is a saccharide; Y is hydrogen, hydroxy, acyloxy, SH, NHR¹⁸, NR¹⁸R¹⁹ or OCOR¹⁹; R², R³, R⁸, are the same or different and independently hydrogen, alkyl, substituted alkyl, or absent, or R⁸ is hydrogen, alkyl, substituted alkyl, or absent, and R² and R³ are the same or different and independently carbon, oxygen, nitrogen, or sulfur taken together with the atom to which they are bonded form a six membered ring that includes A, B, Cₐ, and D resulting in an aromatic carbocycle, heterocycle or homocycle or R⁸ is hydrogen, alkyl, substituted alkyl, or absent, and R² and R³ are the same carbon, oxygen, nitrogen, or sulfur atom taken together with the atoms to which it is bonded form a five membered ring that includes A, B, Cₐ, and D resulting in an aromatic carbocycle, heterocycle or homocycle or R² is hydrogen, alkyl, substituted alkyl, or absent, and R⁸ and R³ are the same or different and independently carbon, oxygen, nitrogen, or sulfur taken together with the atom to which they are bonded form a five membered ring that includes B, Cₐ, and D resulting in an aromatic carbocycle, heterocycle or homocycle or R³ is hydrogen, alkyl, substituted alkyl, or absent, and R² and R⁸ is taken together with the atom to which they are bonded form a three, five or six membered ring of the same or different independently carbon, nitrogen, oxygen, or sulfur atoms that includes A and B resulting in an aromatic carbocycle, heterocycle or homocycle; R⁴, R⁵, R⁶, and R⁷, are the same or different and independently hydrogen, alkyl, substituted alkyl, alkoxy, substituted alkoxy or absent; R⁹, R¹⁰, R¹¹, R¹², R¹⁸, R¹⁹, are the same or different and independently hydrogen, alkyl, or substituted alkyl; and n and m are the same or different and independently 1 to 20, preferably 1 to 2.

In another embodiment, the present invention is a compound having the following formula: 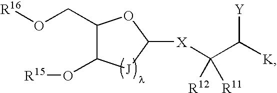
including stereoisomers, prodrugs and pharmaceutically acceptable salts thereof, wherein K is 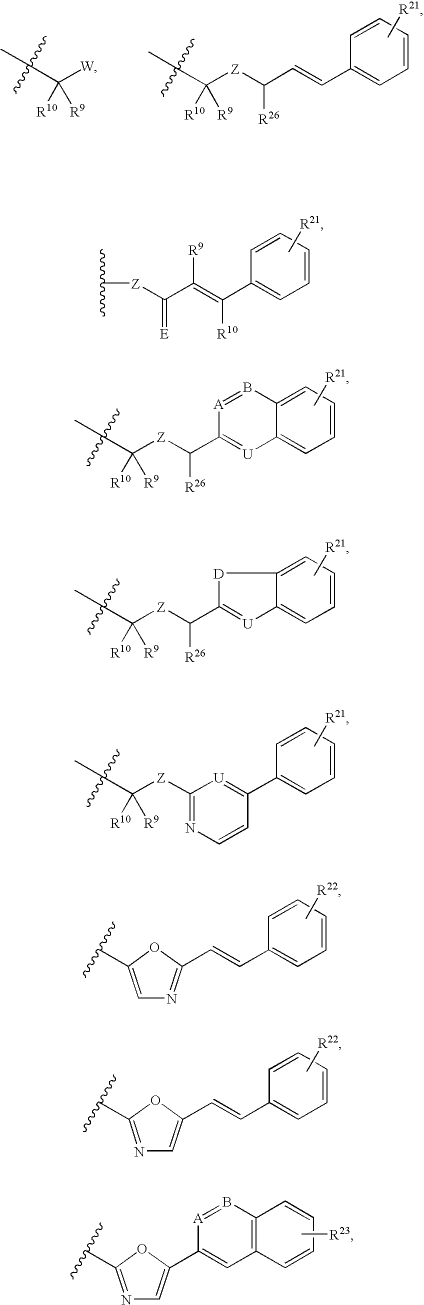 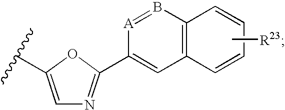
A, B, and U are carbon or nitrogen; E is oxygen or sulfur; D is carbon, nitrogen, oxygen, or sulfur; W is hydroxy, alkoxy, substituted alkoxy, acyloxy or substituted acyloxy; X and Z are the same or different and independently oxygen, sulfur, CH₂, or NR¹⁸; Y is hydrogen, hydroxy, —SH, —NHR¹⁹, —NR¹⁹R²⁰, —CH₂OR²⁴, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl or Y and X taken together with the atoms to which they are bonded form a five or six membered ring where X is nitrogen; J_{λ} is —CHOR¹⁴—, wherein _{λ} denotes an integer from 1 to 3 of J_{λ} groups the same or different and independently sequentially connected together with the atoms that they are attached to from a five, six, or seven membered ring; R²¹, R²², R²³, and R²⁴ are the same or differently and independently hydrogen, halogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ; Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; R⁹, R¹⁰, R¹¹, R¹², R¹⁸R¹⁹ and R²⁰ are the same or different and independently hydrogen or alkyl; and R¹⁴, R¹⁵, and R¹⁶ are the same or different and independently hydrogen, alkyl, substituted alkyl, acyl or substituted acyl.

In another embodiment, the present invention is a compound having the following formula: 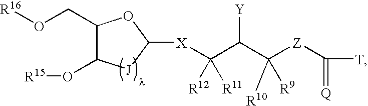
including stereoisomers, prodrugs and pharmaceutically acceptable salts thereof, wherein Q is oxygen, nitrogen, sulfur, two hydrogens, or CH₂; X and Z are the same or different and independently oxygen, sulfur or NR¹⁸; Y is hydrogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl; T is 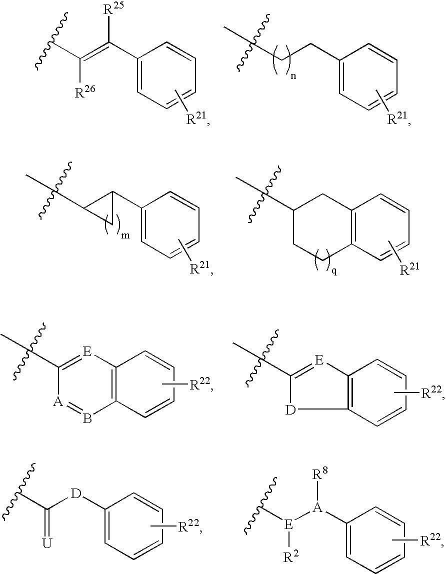
substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ; A, B, and E, is the same or differently and independently carbon or nitrogen, D is carbon, nitrogen, oxygen or sulfur; U is oxygen, sulfur, CH₂, NR¹⁸, or two hydrogens; n is 1 to 6, preferably 1 to 2; m is 1 to 5; q is 1 or 2; J_{λ} is —CHOR¹⁴—, wherein _{λ} denotes an integer from 1 to 3 of J_{λ} groups the same or different and independently sequentially connected together with the atoms that they are attached to from a five, six, or seven membered ring; R⁹, R¹⁰, R¹¹, R¹², and R¹⁸ are the same or different and independently hydrogen or alkyl; R¹⁴, R¹⁵, and R¹⁶ are the same or different and independently hydrogen, alkyl, substituted alkyl, acyl or substituted acyl; R²¹ and R²² are the same or differently and independently hydrogen, halogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ; Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; and R²⁵ and R²⁶ is the same or differently and independently hydrogen or alkyl.

In another embodiment, the present invention is a compound having the following formula: 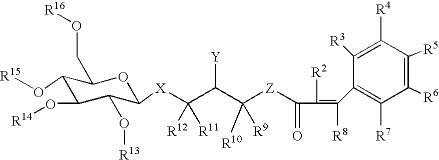
including stereoisomers, prodrugs and pharmaceutically acceptable salts thereof, wherein X and Z are the same or different and independently oxygen, sulfur or NR¹⁸; Y is hydrogen, hydroxy, SH, or NHR¹; R², R⁸ are the same or different and independently hydrogen, alkyl, or substituted alkyl; R³, R⁵, R⁵, R⁶, and R⁷ are the same or different and independently hydrogen, alkyl, substituted alkyl, alkoxy, or substituted alkoxy; R⁹, R¹⁰, R¹¹, R¹², and R¹⁸ are the same or different and independently hydrogen or alkyl; and R¹, R¹³, R¹⁴, R¹⁵, and R¹⁶ are the same or different and independently hydrogen, alkyl, substituted alkyl, acyl or substituted acyl.

In another embodiment, present invention is a compound having the following formula: 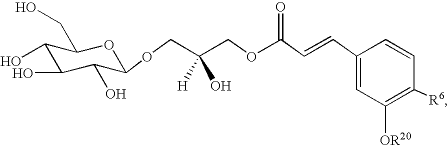
including, prodrugs and pharmaceutically acceptable salts thereof, wherein R⁶ is hydrogen or hydroxy and R²⁰ is hydrogen, alkyl, or acyl.

In another embodiment the present invention contemplates a composition comprising a compound i) functioning to reduce platelet aggregation and ii) having a substituted structure of the following formula:

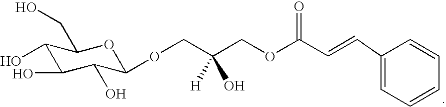

In another embodiment, the present invention contemplates the compounds having the formulas disclosed. In further embodiments, the present invention contemplates compositions comprising the disclosed compounds, wherein the compounds by weight are greater that 1%, 5%, 15%, 20%, 25%, 30%, 40%, 45%, 50%, 55%, 60%, 65%, 70%, 75%, 80%, 85%, 90%, 95%, 98%, 99%, 99.5%, 99.99% of the entire composition.

A chemical group is “absent” means that no atom is connected. This can result when replacing heteroatoms such as oxygen, sulfur, and nitrogen for carbon. The heteroatoms may not attain the same bonding state as carbon. For example, if a carbon atom in a phenyl ring is replaced with a nitrogen atom, the hydrogen atom that was attached to the carbon atom may be absent.

“Acyl” means an —C(═O)alkyl group.

“Acyloxy” means —Oacyl.

“Adverse drug reaction” means any response to a drug that is noxious and unintended and occurs in doses for prophylaxis, diagnosis, or therapy including side effects, toxicity, hypersensitivity, drug interactions, complications, or other idiosyncrasy. Side effects are often adverse symptom produced by a therapeutic serum level of drug produced by its pharmacological effect on unintended organ systems (e.g., blurred vision from anticholinergic antihistamine). A toxic side effect is an adverse symptom or other effect produced by an excessive or prolonged chemical exposure to a drug (e.g., digitalis toxicity, liver toxicity). Hypersensitivities are immune-mediated adverse reactions (e.g., anaphylaxis, allergy). Drug interactions are adverse effects arising from interactions with other drugs, foods or disease states (e.g., warfarin and erythromycin, cisapride and grapefruit, loperamide and Clostridium difficile colitis). Complications are diseases caused by a drug (e.g., NSAID-induced gastric ulcer, estrogen-induced thrombosis). The adverse drug reaction may be mediated by known or unknown mechanisms (e.g., Agranulocytosis associated with chloramphenicol or clozapine). Such adverse drug reaction can be determined by subject observation, assay or animal model well-known in the art.

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. The term “higher alkyl” has the same meaning as alkyl but contains from 2 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cyclic alkyls are also referred to herein as a “homocycles” or “homocyclic rings.” Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkylamino” and “dialkylamino” mean one or two alkyl moiety attached through a nitrogen bridge (i.e., —N-alkyl) such as methylamino, ethylamino, dimethylamino, diethylamino, and the like.

“Alkoxy” means an alkyl moiety attached through an oxygen bridge (i.e., —O-alkyl) such as methoxy, ethoxy, and the like.

“Alkylthio” means an alkyl moiety attached through a sulfur bridge (i.e., —S—alkyl) such as methylthio, ethylthio, and the like.

“Alkylsulfonyl” means an alkyl moiety attached through a sulfonyl bridge (i.e., —SO₂-alkyl) such as methylsulfonyl, ethylsulfonyl, and the like.

“Atherosclerosis” is a gradual process in which plaques (collections) of cholesterol are deposited in the walls of arteries. Cholesterol plaques cause hardening of the arterial walls and narrowing of the inner channel (lumen) of the artery. Arteries that are narrowed by atherosclerosis cannot deliver enough blood to maintain normal function of the parts of the body they supply. For example, atherosclerosis of the arteries in the legs causes reduced blood flow to the legs. Reduced blood flow to the legs can lead to pain in the legs while walking or exercising, leg ulcers, or a delay in the healing of wounds to the legs. Atherosclerosis of the arteries that furnish blood to the brain can lead to vascular dementia (mental deterioration due to gradual death of brain tissue over many years) or stroke (sudden death of brain tissue).

“Aryl” means an aromatic carbocyclic moiety such as phenyl or naphthyl.

“Arylpolyphenol” means an aryl substituted with two or more hydroxyl groups.

“Arylalkyl” means an alkyl having at least one alkyl hydrogen atoms replaced with an aryl moiety, such as benzyl, —(CH₂)₂phenyl, —(CH₂)₃phenyl, —CH(phenyl)₂, and the like.

As used herein, the term “glycerol saccharide component” refers that part of a composition that contains all of glycerol saccharide derivatives in a given composition, including all conformational and stereomeric forms. In preferred embodiments, a given compound (e.g. designated by a structure) makes up a large percentage (e.g. by number of molecules and/or by weight) of the glycerol saccharide component. For example, a given glycerol saccharide derivative may be present in an aqueous composition at a level where 70% of all the glycerol saccharide components are of that given compound, while most of the composition itself is composed of water.

“Enantiomeric excess” (ee) refers to the products that are obtained by a synthesis comprising an enantioselective step, whereby a surplus of one enantiomer in the order of at least about 52% ee is yielded.

A compound “functioning to reduce platelet aggregation” means that the compound is capable of 10% or more of thrombosis inhibition by using the following procedure. Male Sprague-Dawley rats weighing 320-380 g are used after overnight fasting. The rats are administered orally separately with two samples; first trial a control (isotonic saline) and second trial 50 mg/kg of the compound in isotonic saline (a 75 min period occurs between each trial to allow the return of homeostasis). The left jugular vein and the right carotid artery are cannulated with a 4 cm long polyethylene tube (o.d. 1 mm) while being infused with 50 μ/kg i.v. heparin. These catheters are connected to the ends of a 15 cm long polyethylene tube (o.d. 2 mm) containing a 5 cm long suture silk thread (no. 4). Before the silk thread is placed in the polyethylene tube a pre-treatment wet weight is determined (i.e., a pre-thrombus formation weight). After 120 min blood is directed to flow through the shunt for 15 minutes. The silk thread, containing formed thrombi, is gently removed, and a post-treatment wet weight is measured. The formed thrombus wet weight is determined by subtracting the pre-treatment silk wet weight from the post-treatment silk wet weight. The rate of thrombosis inhibition was calculated with the equation as follows: % Thrombosis inhibition=(A−A1)/A×100%, where A is the formed thrombus wet weight of the control group (isotonic saline), and A1 was the newly formed thrombus wet weight of 50 test compound group (50 mg/kg of the compound in isotonic saline pound).

“Halogen” means fluoro, chloro, bromo and iodo.

“Haloalkyl” means an alkyl having at least one hydrogen atom replaced with halogen, such as trifluoromethyl and the like.

“Heart attack” or “myocardial infarction” means the destruction of heart muscle cells due to a lack of oxygen including, but not limited to, episode(s) of heart failure or the stopping of normal heart function because of occlusion of a coronary artery. Coronary arteries are blood vessels that supply the heart muscle with blood and oxygen. Blockage of a coronary artery because of an atherosclerotic plaque deprives the heart muscle of blood and oxygen, causing injury to the heart muscle. Symptoms of a heart attack include pain and pressure in the chest, which often spread to the shoulder, arm, and neck. Heart attacks are generally diagnosed using electrocardiograms and testing blood for enzymes indicating the destruction of heart muscle.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂morpholinyl, and the like.

“Homocycle” (also referred to herein as “homocyclic ring”) means a saturated or unsaturated (but not aromatic) carbocyclic ring containing from 3-7 carbon atoms, such as cyclopropane, cyclobutane, cyclopentane, cyclohexane, cycloheptane, cyclohexene, and the like.

The term “manage” when used in connection with a disease or condition means to provide beneficial effects to a patient being administered with a prophylactic or therapeutic agent, which does not result in a cure of the disease. In certain embodiments, a patient is administered with one or more prophylactic or therapeutic agents to manage a disease so as to prevent the progression or worsening of the disease.

“Methylene” means —CH₂—.

As used herein and unless otherwise indicated, the term “prodrug” means a derivative that can hydrolyze, oxidize, or otherwise react under biological conditions (in vitro or in vivo) to provide an active compound. Examples of prodrugs include, but are not limited to, derivatives and metabolites that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. If present, esterifying any of the carboxylic acid moieties present on the molecule conveniently forms the carboxylate esters.

“Pharmaceutically active derivative” refers to any compound that upon administration to the recipient is capable of providing directly or indirectly, the activity disclosed herein.

“Pharmaceutically acceptable salts or “complexes” refers to salts or complexes of the below-identified compounds that retain the desired biological activity. Examples of such salts include, but are not restricted to acid addition salts formed with inorganic acids (e.g. hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, poly glutamic, acid, naphthalene sulfonic acid, naphthalene disulfonic acid, and poly galacturonic acid. Said compounds can also be administered as pharmaceutically acceptable quaternary salts known by a person skilled in the art, which specifically include the quaternary ammonium salts of the formula —NR,R′,R′⁺Z⁻, wherein R, R′, R″ is independently hydrogen, alkyl, or benzyl, and Z is a counter ion, including chloride, bromide, iodide, alkoxide, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

As used herein, the terms “prevent” and “preventing” include the prevention of the recurrence, spread or onset. It is not intended that the present invention be limited to complete prevention. In some embodiments, the onset is delayed, or the severity of the disease is reduced.

“Saccharide” means a sugar or substituted sugar exemplified by but is not limited to 2,3-dideoxyhex-2-enopyranoside, 2,3-desoxy-2,3-dehydroglucose, 2,3-desoxy-2,3-dehydroglucose diacetate, glucoside, glucoside tetraacetate, mannoside, mannoside tetraacetate, galactoside, galactoside tetraacetate, alloside, alloside tetraacetate, guloside, guloside tetraacetate, idoside, idoside tetraacetate, taloside, taloside tetraacetate, rhamnoside, rhamnoside triacetate, maltoside, maltoside heptaacetate, 2,3-desoxy-2,3-dehydromaltoside, 2,3-desoxy-2,3-dehydromaltoside pentaacetate, 2,3-desoxymaltoside, lactoside, lactoside tetraacetate, 2,3-desoxy-2,3-dehydrolactoside, 2,3-desoxy-2,3-dehydrolactoside pentaacetate, 2,3-desoxylactoside, glucouronate, N-acetylglucosamine, fructose, sorbose, ribose, galactose, glucose, mannose, 2-deoxygalactose, 2-deoxyglucose, maltulose, lactulose, palatinose, leucrose, turanose, lactose, maltose, mannitol, sorbitol, dulcitol, xylitol, erythitol, threitol, adonitol, arabitol, rhamnitol, talitol, 1-aminodulcitol, 1-aminosorbitol, isomaltitol, cellobiitol, lactitol, maltitol, volemitol perseitol, glucoheptitiol, alpha,alpha-glucooctitiol including polysaccharides and polyols (i.e., compounds having a large ratio of primary and secondary protected or unprotected hydroxyl groups where if unprotected have a ratio of hydrogen to carbon atoms near 2:1). Saccharides can be derivatized with molecular arrangements that facilitate production (i.e., contain a protecting group, e.g., acetyl group). Saccharides can be derivatized to form prodrugs.

“Subject” means any animal, preferably a human patient, livestock, or domestic pet.

The term “substituted” as used herein means any of the above groups (i.e., alkyl, aryl, arylalkyl, heteroaryl, heteroarylalkyl, homocycle, heterocycle and/or heterocyclealkyl) wherein at least one hydrogen atom is replaced with a substituent. In the case of an oxo substituent (“═O”), two hydrogen atoms are replaced. When substituted one or more of the above groups are substituted, “substituents” within the context of this invention include halogen, hydroxy, oxo, cyano, nitro, amino, alkylamino, dialkylamino, alkyl, alkoxy, alkylthio, haloalkyl, aryl, arylalkyl, heteroaryl, heteroarylalkyl, heterocycle and heterocyclealkyl, as well as —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ. In addition, the above substituents may be further substituted with one or more of the above substituents, such that the substituent substituted alky, substituted aryl, substituted arylalkyl, substituted heterocycle or substituted heterocyclealkyl. Rₐ and R_{b} in this context may be the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl.

As used herein, the terms “treat” and “treating” are not limited to the case where the subject (e.g. patient) is cured and the disease is eradicated. Rather, the present invention also contemplates treatment that merely reduces symptoms, and/or delays disease progression.

A number of glycerol saccharide derivatives have been disclosed in the following references: Satou et al., Phytochemistry, 41(4), 1225-30 (1996); Del Corral et al., Fitoterapia, 66(4), 371-2 (1995); Mimaki et al., Phytochemistry 31(5), 1753 (1992); Sashida et al., Chemical & Pharmaceutical Bulletin, 39(9), 2362 (1991); Bokern et al., Phytochemistry, 30(10), 3261-5 (1991); Mikaki and Sashida, Phytochemistry, 30(3), 937-40 (1991); Mimaki and Sashida, Chemical & Pharmaceutical Bulletin, 38(11), 3055-9 (1990); Mimaki and Sashida, Phytochemistry, 29(7), 2267-71 (1990); Mimaki and Sashida, Chemical and Pharmaceutical Bulletin, 38(4), 1090-2 (1990); Mimaki et al., Phytochemistry, 28(12) 3453-8 (1989); Shimomur et al., Shoykugaku Zasshi, 43(1) 64-70 (1989); Sashida et al., Chemistry Letters, 5, 897-900 (1989); Shimomura et al., Chemical & Pharmaceutical Bulletin, 36(12), 4841-8 (1988); Shimomura et al., Chemical & Pharmaceutical Bulletin, 36(7), 2430-46 (1988); Shimomura et al., Phytochemistry, 27(2), 451-4 (1988); and Japanese Patent No. 59053497 (1984). In some embodiments of the current invention, the glycerol saccharide derivatives disclosed in these references are discovered to be useful to prevent platelet aggregation and can be use in methods of preventing and treating subjects with heart conditions particularly cardiovascular diseases.

In some embodiments, of the current invention contemplates substituted glycerol saccharide derivatives of the following formula: 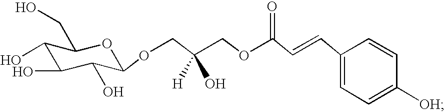
including stereoisomers, prodrugs and pharmaceutically acceptable salts thereof. In some embodiments, the glycerol glucoside derivative has the following formula: 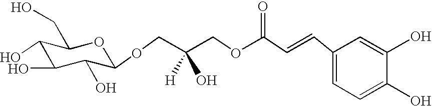
including stereoisomers, prodrugs and pharmaceutically acceptable salts thereof.

In some embodiments, the glycerol glucoside derivative has the following formula: 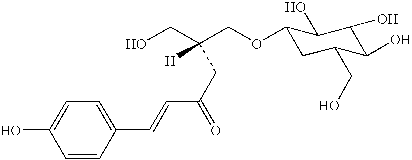
including stereoisomers, prodrugs and pharmaceutically acceptable salts thereof.

In some embodiments, the glycerol glucoside derivative has the following formula: 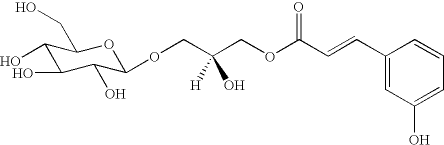
including stereoisomers, prodrugs and pharmaceutically acceptable salts thereof.

In some embodiments, the glycerol glucoside derivative has the following formula: 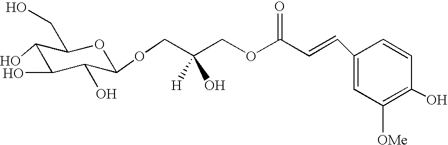
including stereoisomers, prodrugs and pharmaceutically acceptable salts thereof.

In some embodiments, the glycerol glucoside derivative has the following formula: 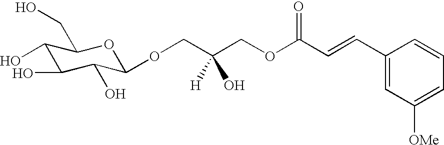
including stereoisomers, prodrugs and pharmaceutically acceptable salts thereof.

In some embodiments, the glycerol glucoside derivative has the following formula: 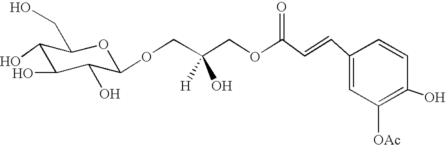
including stereoisomers, prodrugs and pharmaceutically acceptable salts thereof.

In some embodiments, the glycerol glucoside derivative has the following formula: 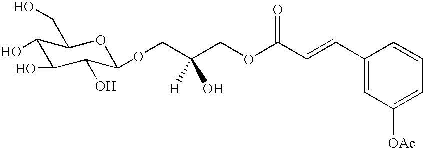
including stereoisomers, prodrugs and pharmaceutically acceptable salts thereof.

In some embodiments, the glycerol glucoside derivative has the following formula: 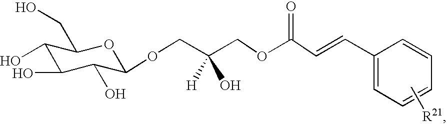
including stereoisomers, prodrugs and pharmaceutically acceptable salts thereof: R²¹ is halogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, as well as —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ, wherein Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl.

In some embodiments, the glycerol glucoside derivative has the following formula: 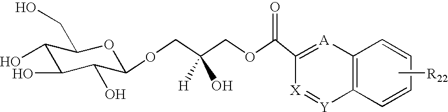
including stereoisomers, prodrugs and pharmaceutically acceptable salts thereof, wherein, R²² is halogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, as well as —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ, wherein Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; and A, X, and Y are the same or different and independently carbon or nitrogen.

In some embodiments, the glycerol glucoside derivative has the following formula: 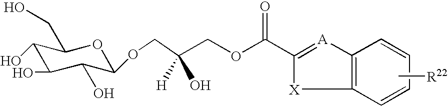
including stereoisomers, prodrugs and pharmaceutically acceptable salts thereof, wherein, R²² is halogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, as well as —NRₐR_{b}, —NR₈C(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ, wherein Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; A is carbon or nitrogen; and X is carbon, nitrogen, oxygen, or sulfur.

In some embodiments, the glycerol glucoside derivative has the following formula: 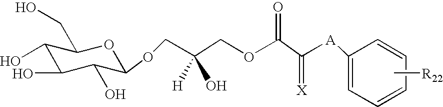
including stereoisomers, prodrugs and pharmaceutically acceptable salts thereof, wherein, R²² is halogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, as well as —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ, wherein Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; A and X are the same or different and independently carbon, nitrogen, oxygen or sulfur.

In some embodiments, the glycerol glucoside derivative has the following formula: 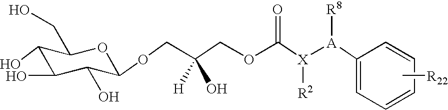
including stereoisomers, prodrugs and pharmaceutically acceptable salts thereof, wherein, and R² and R⁸ is taken together with the atom to which they are bonded form a three, five or six membered ring of the same or independent carbon, nitrogen, oxygen, or sulfur atoms that includes A and X resulting in an aromatic carbocycle, heterocycle or homocycle; R²¹ is halogen, hydroxy, oxo, cyano, nitro, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ, wherein Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; and A and X are the same or different and independently carbon or nitrogen.

In some embodiments, the glycerol glucoside derivative has the following formula: 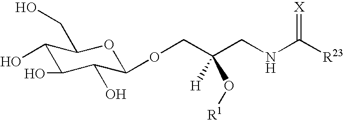
including stereoisomers, prodrugs and pharmaceutically acceptable salts thereof, wherein R¹ is hydrogen, alkyl, substituted alkyl, acyl or substituted acyl; R²³ is hydroxy, cyano, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, as well as —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ, wherein Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; and X is methylene, carbon, nitrogen, oxygen, or sulfur.

In some embodiments, the glycerol glucoside derivative has the following formula: 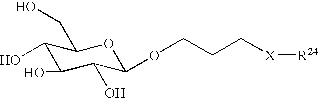
including stereoisomers, prodrugs and pharmaceutically acceptable salts thereof, wherein, R²⁴ is hydroxy, cyano, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, as well as —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ, wherein Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; and X is methylene, carbon, nitrogen, oxygen, or sulfur.

In some embodiments, the glycerol glucoside derivative has the following formula: 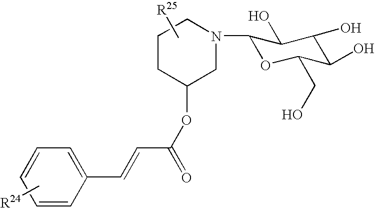
including stereoisomers, prodrugs and pharmaceutically acceptable salts thereof, wherein, R²⁴ and R²⁵ are the same or different and independently hydroxy, cyano, amino, substituted amino, alkylamino, substituted alkyl amino, dialkylamino, substituted dialkylamino, alkyl, substituted alkyl, alkoxy, substituted alkoxy, alkylthio, substituted alkylthiol, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, and heterocyclealkyl, substituted heterocyclealkyl, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ, wherein Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl.

In some embodiments, the combinations of the compounds are administered to prevent or treated diseases disclosed.

In other embodiments, the compounds can be used in anticoagulant therapy in prophylaxis and treatment of venous thrombosis and its extension; in a low-dose regimen for prevention of post-operative deep venous thrombosis and pulmonary embolism in patients undergoing major abdomino-thoracic surgery or who for other reasons are at risk of developing thromboembolic disease; prophylaxis and treatment of pulmonary embolism; atrial fibrillation with embolization; diagnosis and treatment of acute and chronic consumptive coagulopathies (disseminated intravascular coagulation); prevention of clotting in arterial and cardiac surgery; prophylaxis and treatment of peripheral arterial embolism; as an anticoagulant in blood transfusions, extracorporeal circulation, dialysis procedures and in blood samples for laboratory purposes. In further embodiments, the compounds can be used to relieve mild to moderate pain; reduce fever, redness, and swelling; and to help prevent blood from clotting; to relieve discomfort caused by numerous medical problems, including headache, infections, and arthritis; to reduce the risk of a second heart attack or stroke and gout.

Electrocardiogram (ECG/EKG) findings suggestive of myocardial infarction are elevations of the ST segment and changes in the T wave. After a myocardial infarction, changes can often be seen on the ECG called Q waves, representing scarred heart tissue. Cardiac enzymes are proteins from cardiac tissue found in the blood. Disproportional elevation of the MB subtype of the enzyme creatine phosphokinase (CPK) is specific for myocardial injury. Current guidelines are generally in favor of determining troponin isoenzymes I or T, which are thought to rise before permanent injury develops. A positive troponin in the setting of chest pain may accurately predict a high likelihood of a myocardial infarction in the near future.

The compositions comprising the active compound include bulk-drug compositions useful in the manufacture of pharmaceutical compositions (e.g., impure or non-sterile compositions) and pharmaceutical compositions (i.e., compositions that are suitable for administration to a patient) that can be used in the preparation of unit dosage forms. Such compositions optionally comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of the active compound and another therapeutic or prophylactic agent, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the active compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a patient, the pharmaceutically acceptable vehicles are preferably sterile. Water can be the vehicle when the active compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propyleneglycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155).

In a preferred embodiment, the active compound and optionally another therapeutic or prophylactic agent are formulated in accordance with routine procedures as pharmaceutical compositions adapted for intravenous administration to human beings. Typically, the active compound(s) for intravenous administration are solutions in sterile isotonic aqueous buffer. Where necessary, the compositions can also include a solubilizing agent. Compositions for intravenous administration can optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the active compound is to be administered by infusion, it can be dispensed, for example, with an infusion bottle containing sterile pharmaceutical grade water or saline. Where the active compound is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, in tablet or pill form, the compositions can be coated to delay disintegration and absorption in the gastrointestinal tract providing a sustained action over an extended period of time. Selectively permeable membranes surrounding an osmotically active driving compound are also suitable for an orally administered of the active compound. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, and the like. Such vehicles are preferably of pharmaceutical grade.

Further, the effect of the active compound can be delayed or prolonged by proper formulation. For example, a slowly soluble pellet of the active compound can be prepared and incorporated in a tablet or capsule. The technique can be improved by making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound in oily or emulsified vehicles which allow it to disperse only slowly in the serum.

Pharmaceutical compositions for use in accordance with the present invention can be formulated in conventional manner using one or more physiologically acceptable carriers or excipients.

Thus, the compound and optionally another therapeutic or prophylactic agent and their physiologically acceptable salts and solvates can be formulated into pharmaceutical compositions for administration by inhalation or insufflation (either through the mouth or the nose) or oral, parenteral or mucosal (such as buccal, vaginal, rectal, sublingual) administration. In one embodiment, local or systemic parenteral administration is used.

For oral administration, the pharmaceutical compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the pharmaceutical compositions can take the form of tablets or lozenges formulated in conventional manner.

For administration by inhalation, the pharmaceutical compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The pharmaceutical compositions can be formulated for parenteral administration by injection, e.g., by bolus injection or continuous infusion. Formulations for injection can be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The pharmaceutical compositions can also be formulated in rectal compositions such as suppositories or retention enemas, e.g., containing conventional suppository bases such as cocoa butter or other glycerides.

In addition to the formulations described previously, the pharmaceutical compositions can also be formulated as a depot preparation. Such long acting formulations can be administered by implantation (for example subcutaneously or intramuscularly) or by intramuscular injection. Thus, for example, the pharmaceutical compositions can be formulated with suitable polymeric or hydrophobic materials (for example as an emulsion in an acceptable oil) or ion exchange resins, or as sparingly soluble derivatives, for example, as a sparingly soluble salt.

The invention also provides that a pharmaceutical composition is packaged in a hermetically sealed container such as an ampoule or sachet indicating the quantity. In one embodiment, the pharmaceutical composition is supplied as a dry sterilized lyophilized powder or water free concentrate in a hermetically sealed container and can be reconstituted, e.g., with water or saline to the appropriate concentration for administration to a patient.

In other embodiments of the invention, radiation therapy agents such as radioactive isotopes can be given orally as liquids in capsules or as a drink. Radioactive isotopes can also be formulated for intravenous injection. The skilled oncologist can determine the preferred formulation and route of administration.

The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

In certain preferred embodiments, the pack or dispenser contains one or more unit dosage forms containing no more than the recommended dosage formulation as determined in the Physician's Desk Reference (56^{th} ed. 2002, herein incorporated by reference in its entirety).

Methods of administering the active compound and optionally another therapeutic or prophylactic agent include, but are not limited to, parenteral administration (e.g., intradermal, intramuscular, intraperitoneal, intravenous and subcutaneous), epidural, and mucosal (e.g., intranasal, rectal, vaginal, sublingual, buccal or oral routes). In a specific embodiment, the active compound and optionally another prophylactic or therapeutic agents are administered intramuscularly, intravenously, or subcutaneously. The active compound and optionally another prophylactic or therapeutic agent can also be administered by infusion or bolus injection and can be administered together with other biologically active agents. Administration can be local or systemic. The active compound and optionally the prophylactic or therapeutic agent and their physiologically acceptable salts and solvates can also be administered by inhalation or insufflation (either through the mouth or the nose). In a preferred embodiment, local or systemic parenteral administration is used.

In specific embodiments, it can be desirable to administer the active compound locally to the area in need of treatment. This can be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers. In one embodiment, administration can be by direct injection at the site (or former site) of an atherosclerotic plaque tissue.

Pulmonary administration can also be employed, e.g., by use of an inhaler or nebulizer, and formulation with an aerosolizing agent, or via perfusion in a fluorocarbon or synthetic pulmonary surfactant. In certain embodiments, the active compound can be formulated as a suppository, with traditional binders and vehicles such as triglycerides.

In another embodiment, the active compound can be delivered in a vesicle, in particular a liposome.

In yet another embodiment, the active compound can be delivered in a controlled release system. In one embodiment, a pump can be used. In another embodiment, polymeric materials can be used.

The amount of the active compound that is effective in the treatment or prevention of heart conditions can be determined by standard research techniques. For example, the dosage of the active compound which will be effective in the treatment or prevention of heart conditions can be determined by administering the active compound to an animal in a model such as, e.g., the animal models known to those skilled in the art. In addition, in vitro assays can optionally be employed to help identify optimal dosage ranges.

Selection of a particular effective dose can be determined (e.g., via clinical trials) by a skilled artisan based upon the consideration of several factors which will be known to one skilled in the art. Such factors include the disease to be treated or prevented, the symptoms involved, the patient's body mass, the patient's immune status and other factors known by the skilled artisan.

The precise dose to be employed in the formulation will also depend on the route of administration, and the seriousness of the disease-related wasting, and should be decided according to the judgment of the practitioner and each patient's circumstances. Effective doses can be extrapolated from dose-response curves derived from in vitro or animal model test systems.

The dose of the active compound to be administered to a patient, such as a human, is rather widely variable and can be subject to independent judgment. It is often practical to administer the daily dose of the active compound at various hours of the day. However, in any given case, the amount of the active compound administered will depend on such factors as the solubility of the active component, the formulation used, patient condition (such as weight), and/or the route of administration.

The general range of effective amounts of the active compound alone or in combination with another prophylactic or therapeutic agent(s) are from about 0.001 mg/day to about 1000 mg/day, more preferably from about 0.001 mg/day to 750 mg/day, more preferably from about 0.001 mg/day to 500 mg/day, more preferably from about 0.001 mg/day to 250 mg/day, more preferably from about 0.001 mg/day to 100 mg/day, more preferably from about 0.001 mg/day to 75 mg/day, more preferably from about 0.001 mg/day to 50 mg/day, more preferably from about 0.001 mg/day to 25 mg/day, more preferably from about 0.001 mg/day to 10 mg/day, more preferably from about 0.001 mg/day to 1 mg/day. Of course, it is often practical to administer the daily dose of compound in portions, at various hours of the day. However, in any given case, the amount of compound administered will depend on such factors as the solubility of the active component, the formulation used, subject condition (such as weight), and/or the route of administration.

A popular anti-coagulant drug is aspirin. Standardized willow bark is a Chinese herb and is highly standardized source of aspirin (i.e., acetylsalicylic acid). Typical dosage ranges of acetylsalicylic acid include less than 10 mg to 100 mg or more. Particular doses of aspirin include about 5 mg, 10 mg, 20 mg, 40 mg, 50 mg, 60 mg, 80 mg, 100 mg, 150 mg, or 200 mg. Typically, these are daily dosages. Generally, although higher dosages are less preferred because of potential gastric disturbances. Generally, therapeutic dosages range between 40 to 80 mg per day when tolerable by a patient

The invention provides for any method of administrating lower doses of known agents (e.g., aspirin) than previously thought to be useful for the prevention or treatment of heart conditions.

The invention provides a pharmaceutical pack or kit comprising one or more containers containing an active compound and optionally one or more other prophylactic or therapeutic agents useful for the prevention or treatment of heart conditions. The invention also provides a pharmaceutical pack or kit comprising one or more containers containing one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration; or instructions for the composition's use.

The present invention provides kits that can be used in the above methods. In one embodiment, a kit comprises the active compound, in one or more containers, and optionally one or more other prophylactic or therapeutic agents useful for the treatment of heart conditions, in one or more containers.

### EXPERIMENTAL

Adenosine 59-diphosphate (ADP), epinephrine, collagen, bovine serum albumin, prothrombin, thromboplastin, thrombin, hyaluronidase from bovine testis, p-nitrophenyl N-acetyl-b-D-glucosaminide, anti-dinitrophenol (DNP)-IgE, DNP-bovine serum albumin (BSA), Evans blue, hyaluronic acid potassium salts, azelastine, xanthine oxidase (XOD), 2,2-diphenyl-1-picrylhydrzyl (DPPH), and disodium cromoglycate (DSCG) were purchased from Sigma Chemical (U.S.A.). Compounds were isolated from, bulbs of Lilium lanicifolium, the rhizome of Pueraria lobata (family Leguminosae), Ginkgo Biloba, Trigonella foenum-graecum or other known natural sources according to the methods describe below or in Kakegawa H., Matsumoto H., Satoh T., Chem. Pharm. Bull., 33, 642-646 (1985); Kakegawa H., Matsumoto H., Satoh T., Chem. Pharm. Bull., 40, 1439-1442 (1992); Kakegawa H., Matsumoto H., Satoh T., Planta Med., 54, 385-389; and (1988); Kim D.-H., Yu K.-U., Bae E.-A., Han M. J., Biol. Pharm. Bull., 21,628-630 (1998).

### Example 1

### Extraction of Compounds from Bai-He

Bai-He (bulbs of Lilium lanicifolium) was collected from Yixin County, Jiangsu Province, China. The air-dried plant materials (2.5 kg) were cut into small pieces and extracted with 20 L of 80% methanol at room temperature for three times (each 24 hours). After removal of MeOH from the combined filtrate by rotary evaporator, the extract was suspended in H₂O (3.0 L), then partitioned with Et₂O (1.5 L×3) to obtain Et₂O fraction (residue weight 12 g). The remained water layer was further extracted with EtOAc (1.5 L×3) to obtain EtOAc fraction (residue weight 15 g). At last, the water layer was partitioned with n-BuOH (1.5 L×4) yielding n-BuOH fraction (residue weight 50 g).

The n-BuOH fraction was applied to column chromatography over MCI-gel CHP 20P eluted with gradient MeOH in H₂O (100% H₂O to 100% MeOH) to afford 14 fractions. The fraction eluted by 20% to 30% MeOH was further purified on Chromatorex ODS (15˜30% MeOH) to yield compounds 1 (374 mg) and 2 (930 mg). The fraction eluted by 50% MeOH was purified on silica gel (eluent: CHCl₃-MeOH—H₂O) to yield compound 3 (192 mg). Compounds 1, 2 and 3 were identified as regaloside A, regaloside C and acetylated regaloside F, respectively, by comparing their ¹H- and ¹³C-NMR data with those reported in the references.

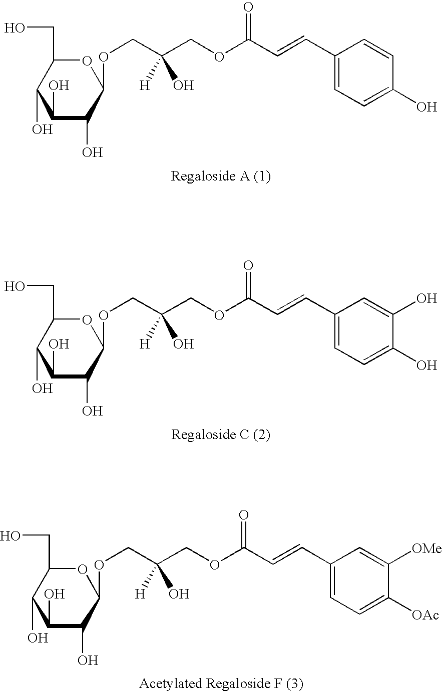

### Example 2

### Assay of in Vitro Antiplatelet Aggregation

Using procedures describe in Example 1 two fractions T2-1 and T2-2 were obtained from Trigonella foenum-graecum after chromatography.

Platelet aggregation was determined using methods and minor variations as described in Choo et al., Biol. Pharm. Bull. 25(10) 1328-1332 (2002) and Born et al., J. Physiol., 168, 178-195 (1963). For example, blood from rats was collected by cardiac puncture into a plastic flask containing 2.2% sodium citrate (1:9 v/v). Platelet-rich plasma (PRP) was prepared by centrifugation of the blood at 120×g for 15 min and further centrifuged at 850×g for 10 min to prepare platelet poor plasma (PPP). The supernatant was pooled and centrifuged at 600×g for 15 min at room temperature. The platelet pellets were washed with modified Tyrode-HEPES buffer (129 mM NaCl, 2.8 mM KCl, 8.9 mM NaHCO3, 0.8 mM MgCl2, 0.8 mM KH2PO4, 2 mM EGTA, 5.6 mM glucose, 10 mM HEPES, 0.35% BSA, pH 7.4) and centrifuged at 600×g for 15 min. Then platelet pellets were gently resuspended in Tyrode-HEPES buffer and used in the experiments.

Platelet aggregation was measured by turbidimetry using a dual-channel Whole Lumini-lonized Calcium Aggregometer (Chrono-Log Co., Ltd., Havertown, Pa., U.S.A.). Rat PRP (300 ml) was incubated at 37° C. for 2 min in the aggregometer with stirring at 1200 rpm and then stimulated with ADP and collagen. The samples or aspirin, as the reference agent, were incubated with PRP for 3 min, followed by the addition of the aggregation agents. Changes in light transmission were recorded for 10 min after stimulation with these agents. Each inhibition rate was obtained from the maximal aggregation induced by the respective agonist at 10 μM or at several concentrations to calculate the IC₅₀ values from the data using a Probit method. Inhibition of the fractions T2-1 and T2-2 at 10 μM were 23.9 and 18.4% respectively. Additional embodiments are shown in Table 1.

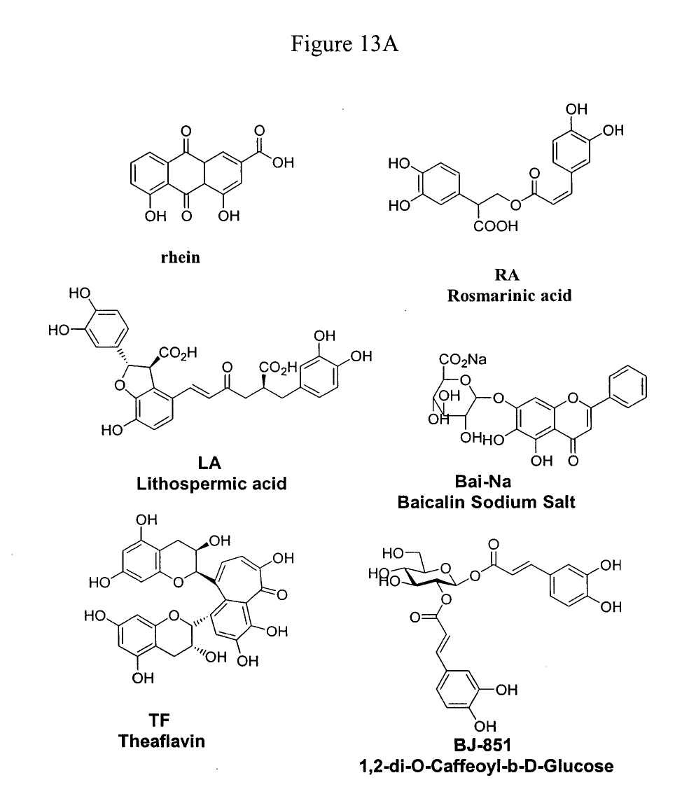

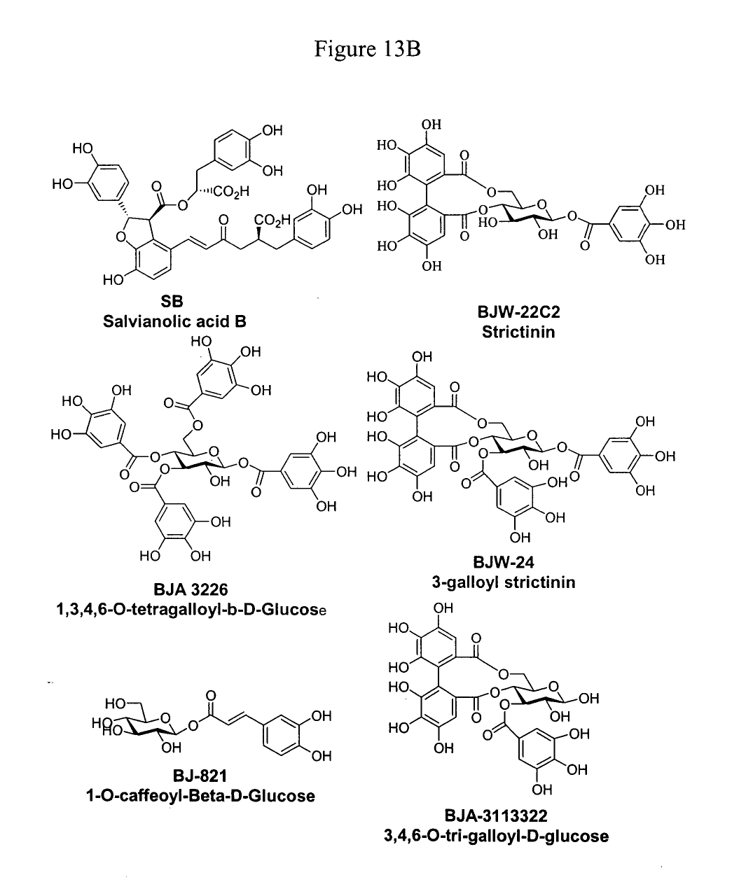

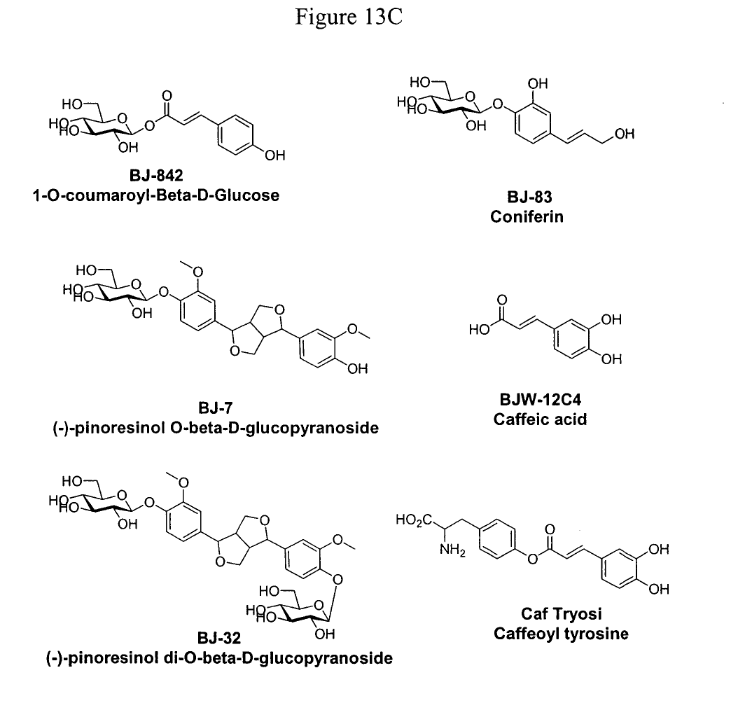

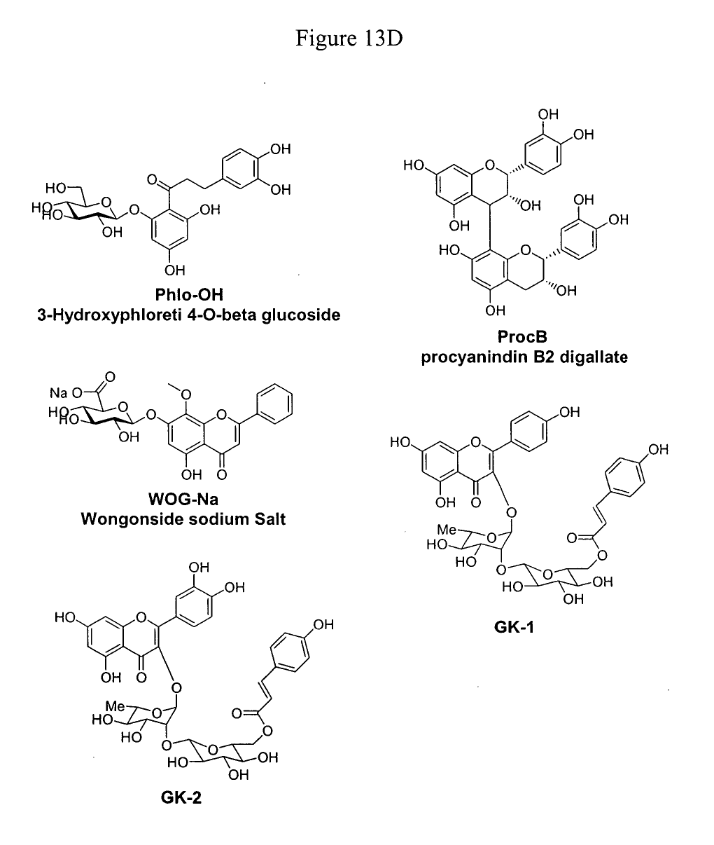

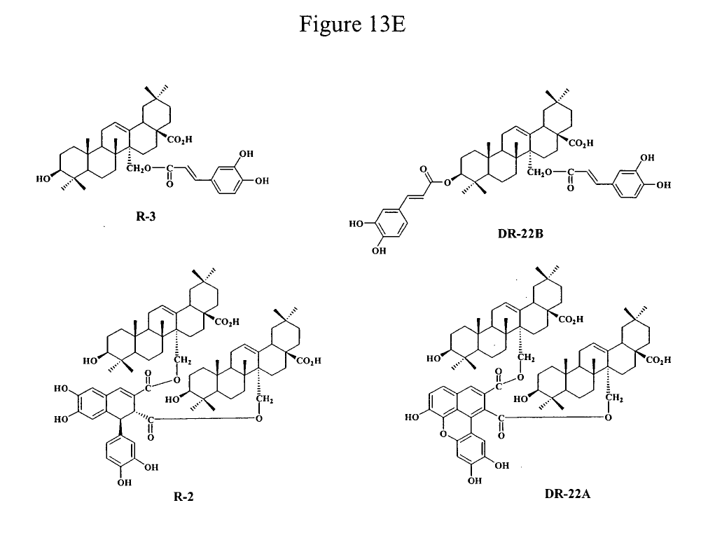

TABLE 1
In Vitro Antiplatelet Aggregation at 10 μM
Structures of Agents shown in FIGS. 13A-E

| Agents | % Inhibition |
|---|---|
| rhein | 1.8 |
| RA | none |
| LA | 0.6 |
| Bai-Na | 12.9 |
| TF | 22.0 |
| BJ-851 | 19.3 |
| SB | 20.1 |
| BJW-22C2 | 0.3 |
| BJA 3226 | 26.6 |
| GK-1 | none |
| GK-2 | 14.9 |
| Phlo-OH | 55.3 |
| ProcB | 15.2 |
| WOG-Na | 112.4 |
| Caf Tryosi | 16.8 |
| BJW-24 | 17.9 |
| BJ-821 | 7.8 |
| BJ-842 | none |
| BJ-83 | 11.1 |
| BJ-7 | 14.1 |
| BJW-32 | 9.21 |
| BJW-12C4 | 5.7 |
| R-3 | 7.1 |
| R-2 | none |
| DR-22B | none |
| DR-22A | 2.4 |

### Example 3

### Animal Model (In Vivo) Assay For Platelet Aggregation Activity

Assays for platelet aggregation activity using arteriovenous shunts were carried out as provided in Umetsu et al., “Effect Of 1-Methyl-2-Mercapto-5(3-Pyridyl)-Imidazole (KC-6141), An Antiaggregation Compound, On Experimental Thrombosis In Rats” Thromb Haemostasis 39:74-83 (1978); and Tang et al., “Anti-Thrombotic Activity Of PDR, A Newly Synthesized L-Arg Derivative, On Three Thrombosis Models In Rats” Thrombosis Research 110:127-133 (2003). Briefly, male Sprague-Dawley rats weighing 320-380 g were used after overnight fasting. The rats were administered orally with: i) a negative control (isotonic saline): ii) a glycerol glucoside derivative (50, 100 mg/kg); and iii) a positive control compound, aspirin (30 mg/kg x 4 times). A 75 min period occurred between each dose to allow the return of homeostasis.

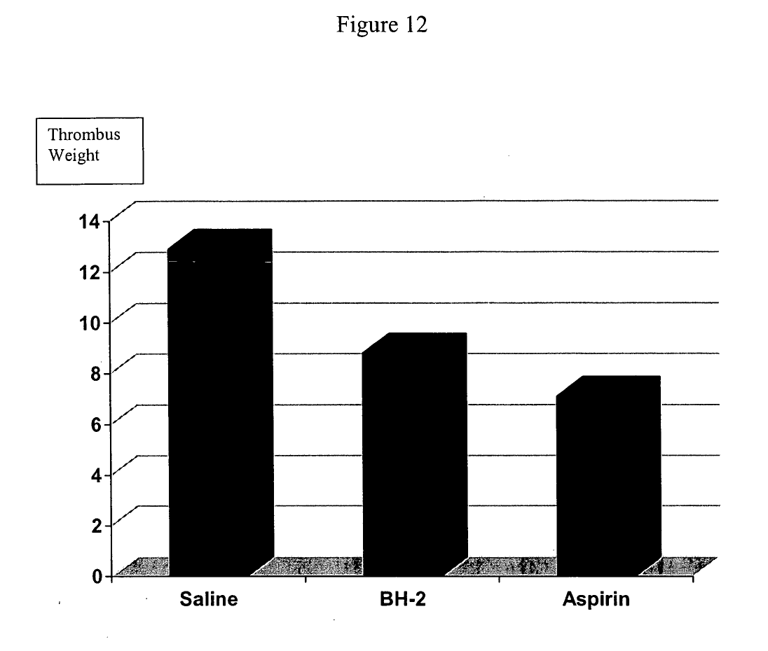

The left jugular vein and the right carotid artery were cannulated with a 4 cm long polyethylene tube (o.d. 1 mm) while being infused with 50 μ/kg i.v. heparin. These catheters were then connected to the ends of a 15 cm long polyethylene tube (o.d. 2 mm) containing a 5 cm long suture silk thread (no. 4). Before the silk thread was placed in the polyethylene tube a pre-treatment wet weight was determined (i.e., a pre-thrombus formation weight). At 120 min after each drug treatment, the blood flow through the shunt was measured for 15 minutes. The silk thread, now containing newly formed thrombi, was gently removed, and a post-treatment wet weight was measured immediately. The newly formed thrombus wet weight was determined by subtracting the pre-treatment silk wet weight from the post-treatment silk wet weight. Thrombus weight for saline was 12.9 mg, for BH-2 (Regaloside C (2)) was 8.8 mg, and for aspirin was 7.1 mg (see FIG. 12).

The rate of thrombosis inhibition was calculated with the equation as follows:
% Thrombosis inhibition=(A−A1)/A×100%

Where A was the newly formed thrombus wet weight of the control group (i.e., Saline), and A1 was the newly formed thrombus wet weight of the test derivative or positive control compound (i.e., aspirin). Thus, the thrombosis inhibition for BH-2 is 31.7% [i.e., (12.9-8.8)/12.9×100].

### Example 4

### A Method for Preparation of Glycerol Glucoside Cinnamate Ester Derivatives

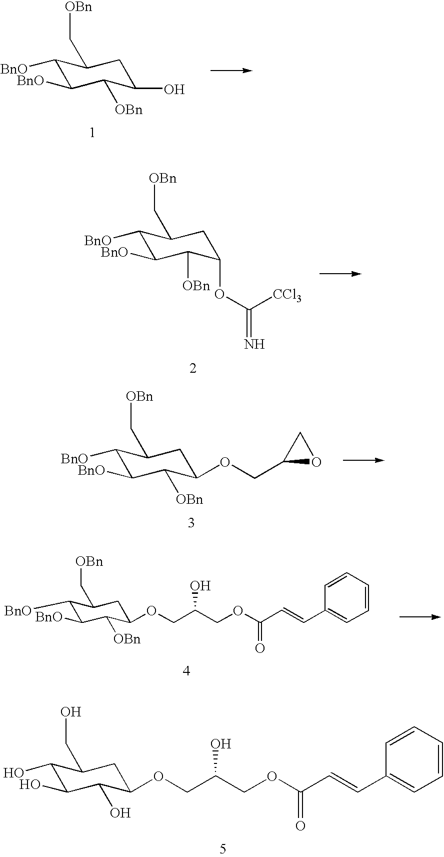

Dichloromethane is added to protected glucose 1 and powdered 4 Å molecular sieves, followed by freshly distilled trichloroacetonitrile. After stirring for one hour, cesium carbonate is added under an inert atmosphere. Triethylamine is added and after 1 hour solvent was removed under reduced pressure. The resulting compound is purified by flash chromatography to afford trichloroacetimidate 2. Compound 2 and ((S)-oxiran-2-yl)methanol are coevaporated with toluene and then dissolved in anhydrous dichloromethane before freshly activated powdered molecular sieves is added. TBSOTF (t-butyldimethylsilyl triflate) is added drop wise at −78° C., and the mixture is then warmed to room temperature for over 2.5 hours. Triethylamine is added, the mixture is filtered through a pad of celite, and the solvent is removed under reduced pressure. Flash chromatography on silica gel affords 3. Cinnamic acid is dissolved in absolute ethanol and sodium ethoxide is added at room temperature. The resulting mixture is stirred at room temperature for half an hour and then 3 in ethanol was added in. The resulting mixture is refluxed for 4 hours and the solvent was removed under reduced pressure. Flash chromatography on silica gel affords 4. Compound 4 is dissolved in ethyl acetae/95% ethanol (2:1) and 10% Pd/C is added under inert environment. The resulting mixture is stirred with a hydrogen balloon for 2 days at room temperature. The mixture is filtered through a pad of celite, and the solvent is removed under reduced pressure. Flash chromatography on silica gel affords 5.

### Example 5

### Second Method for Preparation of Glycerol Glucoside Cinnamate Ester Derivative

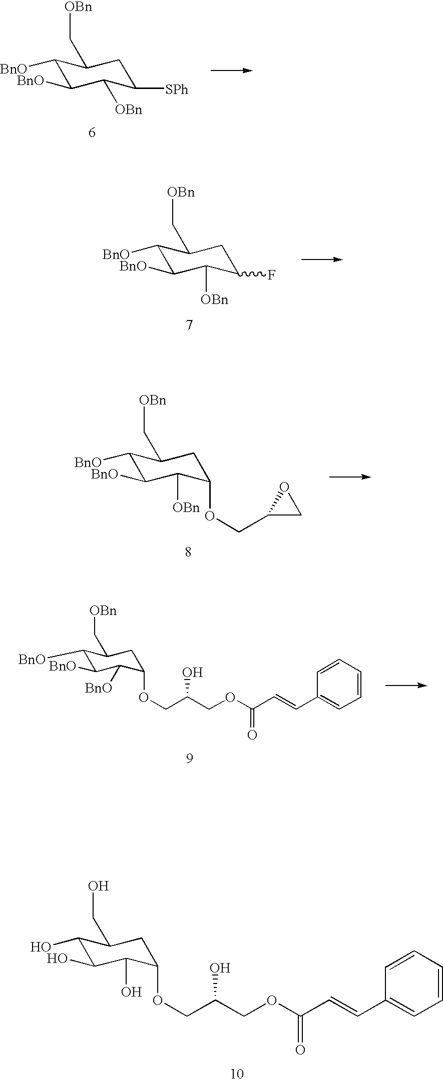

To a solution of 6 and NBS (N-bromosuccinimide) in dichloromethane at 0° C. is added diethylaminosulfur trifluoride. After stirring for 1.8 hours and warming to room temperature, the reaction is poured into a mixture of ice and saturated aqueous sodium bicarbonate, diluted with EtOAc, washed with saturated sodium bicarbonate and brine, and dried. The solvent is removed under reduced pressure. The resulting compound is purified by flash chromatography on silica gel to afford 7. To a mixture of 7 and ((S)-oxiran-2-yl)methanol, hafnocene dichloride (Bis(cyclopentadienyl)hafnium dichloride), 2,6-di-tert-butyl-4-methylpyridine, silver triflate, and 4 Å molecular sieves (powdered) at 0° C. is added dichloromethane. After 40 minutes, the reaction is quenched with saturated sodium bicarbonate, diluted with EtOAc, and filtered through a layer of Celite, rinsing with EtOAc. The filtrate is washed with saturated aqueous sodium bicarbonate and brine. The solvent is removed under reduced pressure. The resulting compound is purified by flash chromatography on silica gel to afford 8.

I claim

1. A method of treating or preventing cardiovascular disease comprising administering to a subject a composition comprising a compound i) functioning to prevent platelet aggregation and ii) having a substituted or unsubstituted formula selected from the group consisting of:

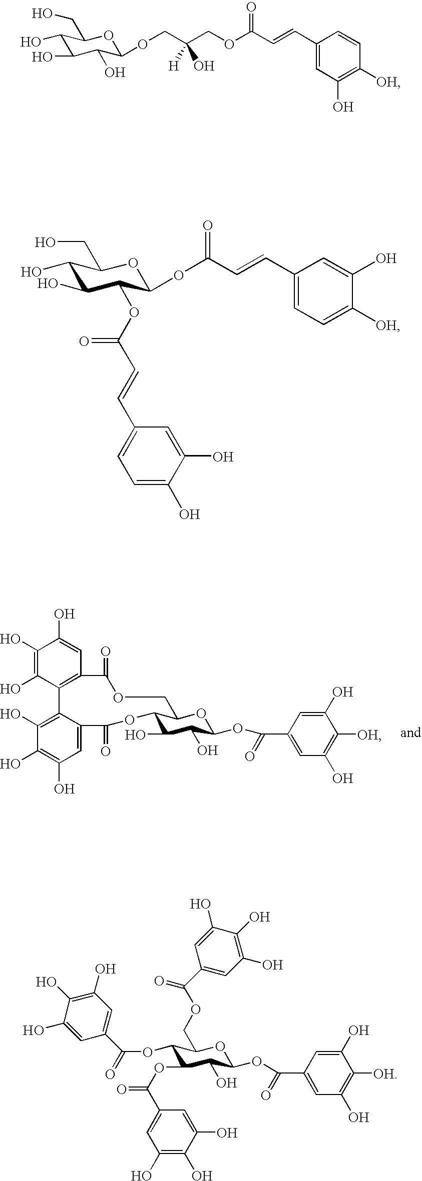

2. A method of treating or preventing cardiovascular disease comprising administering to a subject a composition comprising a compound i) functioning to prevent platelet aggregation and ii) having the following substituted or unsubstituted formula: 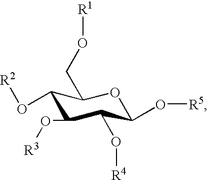
wherein
R¹, R², R³, R⁴ and R⁵ are the same or different and independently are selected from the group consisting of: hydrogen, 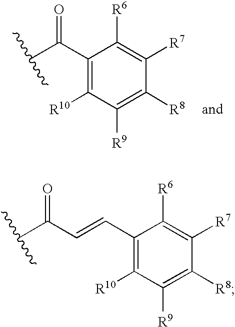
wherein
R⁶, R⁷, R⁸, R⁹ and R¹⁰ are the same or different and independently hydrogen or hydroxyl; or
R³, R⁴ and R⁵ are the same or different and independently are selected from the group consisting of: hydrogen and 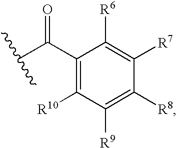
and
R¹ and R² are 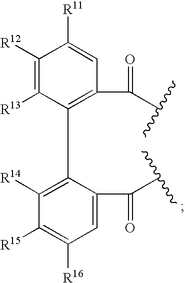
provided that of the group R¹, R², R³, R⁴ and R⁵ no more than three of the members are hydrogen.

3. A method of treating or preventing cardiovascular disease comprising administering to a subject a composition comprising a compound i) functioning to prevent platelet aggregation and ii) having a substituted or unsubstituted formula selected from the group consisting of:

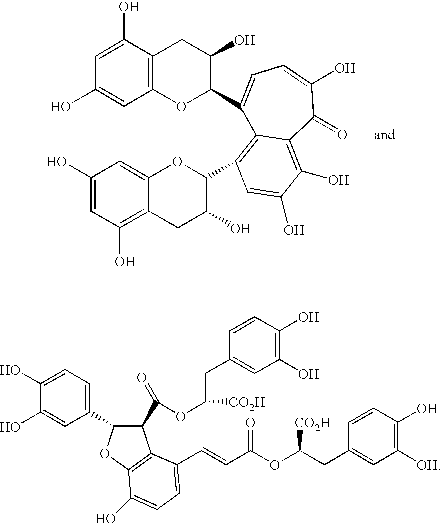

4. A method of treating or preventing cardiovascular disease comprising administering to a subject a composition comprising a compound
i) functioning to reduce platelet aggregation and
ii) having a substituted structure of the following formula: [chemical structure]
